(19) United States
(12) Patent Application Publication
(10) Pub. No.: US 2011/0159586 A1
(43) Pub. Date: Jun. 30, 2011

(54) COMPOSITIONS AND METHODS FOR MODULATING GENE EXPRESSION USING ASYMMETRICALLY-ACTIVE PRECURSOR POLYNUCLEOTIDES

(75) Inventor: Todd Hauser, Seattle, WA (US)

(73) Assignee: HALO-BIO RNAI THERAPEUTICS, INC., Seattle, WA (US)

(21) Appl. No.: 12/746,153

(22) PCT Filed: Dec. 8, 2008
(86) PCT No.: PCT/US08/85985

§ 371 Date: Feb. 28, 2011

Related U.S. Application Data
(60) Provisional application No. 61/012,173, filed on Dec. 7, 2007.

(51) Int. Cl. C12N 5/071; C12N 15/63; C12N 5/10; C12N 1/00; C07H 21/00; C07H 21/02; C07H 21/04

(52) U.S. Cl. 435/375; 435/320.1; 435/325; 435/419; 435/243; 536/24.5

(57) ABSTRACT

The present invention is directed to novel nucleic acid molecules which include a region complementary to a target gene and one or more self-complementary regions, and the use of such nucleic acid molecules and compositions comprising the same to modulate gene expression and treat a variety of diseases and infections.

### CROSS-REFERENCES TO RELATED APPLICATION

This application is a U.S. national stage application filed under 35 U.S.C. §371 of International Patent Application PCT/US2008/085985, accorded an international filing date of Dec. 8, 2008, which claims the benefit under 35 U.S.C. §119(e) of U.S. Provisional Patent Application No. 61/012,173 filed Dec. 7, 2007, where this provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 430157_{—}402USPC_SEQUENCE_LISTING.txt. The text file is 19 KB, was created on Jun. 3, 2010, and is being submitted electronically via EFS-Web.

### BACKGROUND OF THE INVENTION

1. Field of the Invention

The present invention relates generally to structured precursor polynucleotides that produce chemically asymmetric products and guide endonucleases to their respective target molecules, as well as methods of using the same to modulate gene expression and to treat or reduce disease.

2. Description of the Related Art

The phenomenon of gene silencing, or inhibiting the expression of a gene, holds significant promise for therapeutic and diagnostic purposes, as well as for the study of gene function itself. Examples of this phenomenon include antisense technology and dsRNA forms of posttranscriptional gene silencing (PTGS) which has become popular in the form of RNA interference (RNAi), including those methods employing short interfering RNA (siRNA) or short hairpin RNA (shRNA).

Antisense strategies for gene silencing have attracted much attention in recent years. The underlying concept is simple yet, in principle, effective: antisense nucleic acids (NA) base pair with a target RNA resulting in inactivation. Target RNA recognition by antisense RNA or DNA can be considered a hybridization reaction. Since the target is bound through sequence complementarity, this implies that an appropriate choice of antisense NA should ensure high specificity. Inactivation of the targeted RNA can occur via different pathways, dependent on the nature of the antisense NA (either modified or unmodified DNA or RNA or mixtures thereof) and on the properties of the biological system in which inhibition is to occur.

RNAi-based gene suppression is a widely accepted method in which dsRNA as a long RNA duplex, a 19-24 nucleotide duplex, or as a short-hairpin dsRNA (shRNA) duplex are involved in gene modulation by involving enzyme and/or protein complex machinery. The long RNA duplex and the shRNA duplex are pre-cursors that are processed into siRNA by the endoribonuclease described as Dicer. The processed siRNA or directly introduced siRNA is believed to join the protein complex RISC for guidance to a complementary mRNA that is cleaved by the RISC/siRNA complex.

However, many problems persist in the development of effective antisense and RNAi technologies. For example, typical siRNA is based on symmetrical dsRNA and can load into RISC complexes from either end. This loading can determine which strand acts as a guide for RNA/endonuclease complex as it compliments to its target. Thus, the siRNA used in RNAi has proven to result in significant off-target suppression due to either strand guiding cleaving complexes potential involvement in endogenous regulatory pathways. Thermodynamics have been suggested as a control for this process, but many empirical results show effective siRNA with thermodynamics contrary to claims. For synthetic siRNA, it has become common practice to chemically modify one strand to deactivate its function, but there remains no solution for constitutively expressed RNAi molecules to achieve the specificity of chemically modified variants.

DNA antisense oligonucleotides exhibit only short-term effectiveness and are usually toxic at the doses required. Similarly, the use of antisense RNAs has also proved ineffective due to stability problems. Various methods have been employed in attempts to improve antisense stability by reducing nuclease sensitivity and chemical modifications to siRNA have been utilized. These include modifying the normal phosphodiester backbone, e.g., using phosphorothioates or methyl phosphonates, incorporating 2′-OMe-nucleotides, using peptide nucleic acids (PNAs) and using 3′-terminal caps, such as 3′-aminopropyl modifications or 3′-3′ terminal linkages. However, these methods can be expensive and require additional steps. In addition, the use of non-naturally occurring nucleotides and modifications precludes the ability to express the antisense or siRNA sequences in vivo, thereby requiring them to be synthesized and administered afterwards.

Consequently, there remains a need for effective and sustained methods and compositions for the targeted, directed inhibition of gene function in vitro and in vivo, particularly in cells of higher vertebrates, including improved antisense RNAs having increased stability and increased specificity of dsRNA based approaches.

### BRIEF SUMMARY OF THE INVENTION

The present invention relates generally to structured, precursor polynucleotides that produce chemically asymmetric products, and which guide endonucleases to their respective target molecules, and methods of using the same to modulate gene expression and to treat or reduce disease.

In one embodiment, the present invention includes an isolated structured self-forming precursor polynucleotide comprising a region having a sequence complementary to a target gene sequence and one or more self-complementary regions. In particular embodiments, the oligonucleotide comprises two or more self-complementary regions. In particular precursor compositions, the self-complementary regions are located at both ends of the polynucleotide.

In certain embodiments, self-forming precursor polynucleotides of the present invention comprise RNA, DNA, or peptide nucleic acids, or any combination thereof.

In additional embodiments, a self-forming precursor polynucleotide further comprises a second region comprising a sequence that is non-complementary, semi-complementary, or fully complementary to a target gene sequence and non-complementary to a self-complementary region, wherein said second region is located between the self-complementary region and the sequence complementary to a target gene sequence.

In particular embodiments, a self-complementary region comprises a stem-loop structure.

In related embodiments, a self-complementary region is not complementary to the sequence complementary to a target gene sequence.

In related embodiments, a self-complementary region is fully complementary to the part of the sequence complementary to a target gene sequence.

In further related embodiments, wherein the polynucleotide comprises two self-complementary regions, the two self-complementary regions do not complement each other.

In particular embodiments, the sequence complementary to a target gene sequence comprises at least 17 nucleotides, or 17 to 30 nucleotides.

In other embodiments, the self-complementary region comprises at least 5 nucleotides, at least 12 nucleotides, at least 24 nucleotides, or 12 to 54 nucleotides.

In further embodiments, a loop region of a stem-loop structure comprises at least 1 nucleotide. In other embodiments, the loop region comprises at least 2, at least 3, at least 4, at least 5, at least 6, or at least 8 nucleotides.

In further embodiments, a loop region of a stem-loop structure is comprised of a specific tetra-loop sequence NGNN or AAGU.

In another embodiment, the present invention includes an array comprising a plurality of self-forming precursor polynucleotides of the present invention.

In a further embodiment, the present invention includes an expression vector capable of expressing a self-forming precursor polynucleotide of the present invention. In various embodiments, the expression vector is a constitutive or an inducible vector.

The present invention further includes a composition comprising a physiologically acceptable carrier and a self-forming precursor polynucleotide of the present invention.

In other embodiments, the present invention provides a method for reducing the expression of a gene, comprising introducing self-forming precursor oligonucleotides of the present invention into a cell. In various embodiments, the cell is plant, animal, protozoan, viral, bacterial, or fungal. In one embodiment, the cell is mammalian.

In some embodiments, the polynucleotide is introduced directly into the cell, while in other embodiments, the polynucleotide is introduced extracellularly by a means sufficient to deliver the isolated polynucleotide into the cell.

In another embodiment, the present invention includes a method for treating a disease or infection, comprising introducing a self-forming precursor polynucleotide of the present invention into a cell, wherein over expression of the targeted gene is associated with the disease. In one embodiment, the disease is a cancer.

The present invention further provides a method of treating an infection in a patient, comprising introducing into the patient a self-forming precursor polynucleotide of the present invention, wherein the isolated polynucleotide mediates entry, replication, integration, transmission, or maintenance of an infective agent.

In yet another related embodiment, the present invention provides a method for identifying a function of a gene, comprising introducing into a cell a self-forming precursor oligonucleotides of the present invention, wherein the polynucleotide inhibits expression of the gene, and determining the effect of the introduction of the polynucleotide on a characteristic of the cell, thereby determining the function of the targeted gene. In one embodiment, the method is performed using high throughput screening.

In a further embodiment, the present invention provides a method of designing a polynucleotide sequence comprising one or more self-complementary regions for the regulation of expression of a target gene, comprising: (a) selecting a first sequence 17 to 30 nucleotides in length and complementary to a target gene; (b) selecting one or more additional sequences 12 to 54 nucleotides in length, which comprises self-complementary regions and which are not fully-complementary to the first sequence; and optionally (c) defining the sequence motif in (b) to be complementary, non-complementary, or replicate a gene sequence which are non-complementary to the sequence selected in step (a).

In another embodiment, a self-forming precursor polynucleotide of the present invention exhibits an increased half-life in vivo, as compared to the same polynucleotide lacking the one or more self-complementary regions.

In a further related embodiment, the present invention provides a method for treating a disease (e.g., a disease associated with a mutated gene or gene), comprising introducing a self-forming precursor polynucleotide into a cell, wherein the targeted gene comprises one or more mutations as compared to a corresponding wild-type gene.

Similarly, in a related embodiment, the invention includes a method of modulating the expressing of a mutated gene in a cell, comprising introducing a self-forming precursor polynucleotide into a cell, wherein said target RNA sequence comprises a region of said mutated gene. In one specific embodiment, the mutated gene is associated with a tumor. In another embodiment, the mutated gene is a gene expressed from a gene encoding a mutant p53 polypeptide. In another embodiment, the gene is bacterial (MRSA). In another embodiment, the gene is viral.

In one particular embodiment, the present invention provides an isolated, self-forming precursor polynucleotide, comprising: (a) a targeting region comprising a polynucleotide sequence complementary to a region of a target gene sequence; (b) a first self-complementary region; and (c) a second self-complementary region, wherein the first and second self-complementary regions are located one at each end of the targeting region and both self-complementary regions form stem-loop structures, wherein the first self-complementary region is capable of being cleaved by a RNase III endoribonuclease that is not a class IV DICER endoribonuclease, and wherein both self-complementary regions comprise a nucleotide sequence that is complementary to a region of the target gene sequence, but wherein a portion of the target sequence present in the targeting region does not have a complementary sequence in either of the self-complementary regions. In certain embodiments, the polynucleotide sequence complementary to the region of the target gene sequence of (a) consists of about 17 to about 30 nucleotides in length.

In a further related embodiment, the present invention provides an isolated polynucleotide comprising from 5′ to 3′: (a) a first self-complementary region capable of forming a stem-loop structure; (b) a targeting region comprising a polynucleotide sequence reverse complementary to a region of a target gene sequence; and (c) a second self-complementary region capable of forming a stem-loop structure, wherein at least one of the first or second self-complementary regions is capable of being cleaved by a RNase III endoribonuclease that is not a class IV DICER endoribonuclease, and wherein both the first and second self-complementary regions comprise a nucleotide sequence that is complementary to a region of the target gene sequence, but wherein a portion of the target sequence present in the targeting region does not have a complementary sequence in either of the self-complementary regions. In one embodiment, the polynucleotide sequence reverse complementary to the region of the target gene sequence of (b) consists of about 17 to about 30 nucleotides in length.

In one embodiment of self-forming precursor polynucleotides of the present invention, both the first and second self-complementary regions form stem-loop structures capable of being cleaved by a RNase III endoribonuclease that is not a class IV DICER endoribonuclease. In various embodiment, the self-complementary regions that form stem-loop structures capable of being cleaved by a RNase III endoribonuclease that is not a class IV DICER endoribonuclease are about 28 to about 54 nucleotides in length. In particular embodiments, the stem-loop structure of the self-complementary regions capable of being cleaved by a RNase III endoribonuclease that is not a class IV DICER endoribonuclease comprise loops consisting of 2-6 nucleotides. In one embodiment, the stem-loop structure of the self-complementary regions capable of being cleaved by a RNase III endoribonuclease that is not a class IV DICER endoribonuclease comprise tetraloops consisting of a nucleotide sequence selected from NGNN and AAGU.

In various embodiments of the precursor polynucleotides of the present invention, the resulting asymmetric siRNA produced following processing is capable of loading RISC and/or the RISC complex.

In particular embodiments, the loop of a stem-loop structure may comprise an antisense sequence to a target gene, which may be the same or different that the gene targeted by the central targeting region.

In certain embodiments of isolated, self-forming precursor polynucleotides of the present invention, as well as the polynucleotides described immediately above, the stem-loop structure of the second self-complementary region comprises a loop structure of 5 nucleotides to about 14 nucleotides and is resistant to cleavage by a RNase III endoribonuclease that is not a class IV DICER endoribonuclease. In particular embodiment, the second self-complementary region comprises about 5-14 complementary base pairs that form a helical structure. In one embodiment, the second self-complementary region is capable of being cleaved by a class IV DICER endoribonuclease. In particular embodiments, the second self-complementary region is about 9 to about 20 nucleotides in length.

According to certain embodiments, the isolated, self-forming precursor polynucleotide or the polynucleotide described directly above is capable of being processed in a cell to form a biologically active asymmetric siRNA or miRNA polynucleotide. In one embodiment, the processing is by a RNase III endoribonuclease that is not a class IV DICER endoribonuclease. In another embodiment, the processing is by both a RNase III endoribonuclease that is not a class IV DICER and a class IV DICER endoribonuclease. In particular embodiment, the RNase III endoribonuclease that is not a class IV DICER endoribonuclease is selected from Rnt1 and Pac1.

In one embodiment, an isolated, self-forming precursor polynucleotide or other polynucleotide of the present invention is RNA.

In another embodiment, a polynucleotide of the present invention is double-stranded DNA.

In related embodiments, the present invention includes an expression vector capable of producing a polynucleotide of the present invention, including the self-forming precursor polynucleotide described herein. In one embodiment, the expression vector comprises a promoter and a double-stranded DNA polynucleotide of the present invention.

In a further embodiment, the present invention includes a host cell comprising an expression vector of the present invention.

In related embodiments, the present invention also includes a method of inhibiting or reducing expression of a target gene in a cell, comprising introducing a polynucleotide of the present invention into the cell, thereby inhibiting or reducing expression of the target gene in the cell. In one embodiment, the polynucleotide is processed in the cell by a RNase III endoribonuclease that is not a class IV DICER endoribonuclease to form a biologically active asymmetric siRNA or miRNA polynucleotide. In another related embodiment, the polynucleotide is processed in the cell by a RNase III endoribonuclease that is not a class IV DICER endoribonuclease and a class IV DICER endoribonuclease to form a biologically active asymmetric siRNA or miRNA polynucleotide.

In a further related embodiment, the present invention provides a method of inhibiting or reducing expression of a target gene in a cell, comprising introducing an expression vector of the present invention into the cell, thereby inhibiting or reducing expression of the target gene in the cell. In one embodiment, the polynucleotide produced by the expression vector is processed in the cell by a RNase III endoribonuclease that is not a class IV DICER endoribonuclease to form a biologically active asymmetric siRNA or miRNA polynucleotide. In another embodiment, the polynucleotide produced by the expression vector is processed in the cell by a RNase III endoribonuclease that is not a class IV DICER endoribonuclease and a class IV DICER endonuclease to form a biologically active asymmetric siRNA or miRNA polynucleotide.

### BRIEF DESCRIPTION OF THE SEVERAL VIEWS OF THE DRAWING(S)

FIGS. I through VII provide illustrative diagrams of various exemplary polynucleotide structures of the present invention.

FIG. I shows an exemplary self-forming precursor polynucleotide in which the target gene sequence (A) is flanked at the 3′ end by a first self-complementary region that contains a tetraloop sequence (C), and which is cleavable by a Class I, Class II, and/or Class RNase III that is not a Class IV DICER enzyme. FIG. I (E) indicates the 11/13 (x) or 14/16 (x) nucleotide cleavage site of the stem-loop structure by RNase III in pre-processing. The target gene sequence of the polynucleotide in FIG. I is flanked at the 5′ end by a second self-complementary region (B), which is capped by a loop structure (D) that contains greater than 4 nucleotides (here, 8 nucleotides), and which is not only resistant to certain RNase III endoribonucleases, such as Rnt1, but can be processed by DICER subsequent to the processing of the self-complementary region.

FIG. II shows the reverse of the exemplary self-forming precursor polynucleotide of FIG. I. In FIG. I, (H) indicates the location of a proximal box, preferably GAAA and typically excluding a Guanine at position 2, and (I) indicates the location of an distal box of particular nucleotide content. These boxes represent double-stranded RNA binding domains.

FIG. III shows an exemplary self-forming precursor polynucleotide, in which the target gene sequence is flanked on each side by a self-complementary region that contains a tetraloop sequence, and which is first cleavable by a Class I, Class II, and/or Class RNase III that is not a Class IV DICER enzyme.

FIG. IV shows an exemplary self-forming precursor polynucleotide similar to that of FIG. I, in which (F) indicates a tetraloop stem-loop structure duplicated from a UTR region of a targeted gene.

FIG. V shows an exemplary self-forming precursor polynucleotide similar to that of FIG. I, in which (G) indicates a stem-loop sequence that is complementary to the stem-loop sequence of a targeted gene.

FIG. VI illustrates the precursor biogenesis of an exemplary self-forming precursor polynucleotide similar to that of FIG. I. FIG. VI(A) shows the full-length RNase III->DICER->RNA-induced Silencing Complex (RISC) precursor structure. FIG. VI(B) shows the polynucleotide produced by the first step of biogenesis, in which a Class I, Class II, or Class III RNase III endoribonuclease cleaves the 3′ end to produce an asymmetric shRNA-like product that is ready for DICER processing. FIG. VI(C) shows the DICER-processed end product, which represents an active asymmetric siRNA polynucleotide that is capable of activating the RISC complex to target multiple mRNAs.

FIG. VII illustrates the precursor biogenesis of an exemplary self-forming precursor polynucleotide similar to that of FIG. III. FIG. VII(A) shows the full-length, direct-to-RISC precursor structure, prior to processing by a Class I, Class III, or Class III RNase III endoribonuclease. FIG. VII (B) shows the active asymmetric siRNA polynucleotide produced by Class I, Class II, or Class III RNase III-mediated cleavage of the tetraloop containing self-complementary regions.

### DETAILED DESCRIPTION OF THE INVENTION

The present invention provides novel compositions and methods for inhibiting the expression of a target gene in prokaryotes and eukaryotes in vivo and in vitro.

Embodiments of the present invention are based, in part, on the surprising discovery that endogenous nucleases can be used to process precursor RNAi molecules in vivo to produce asymmetric RNAi molecules capable of directing endonucleases to target genes, thus selectively reducing target gene expression with little or no off-target suppression activity. For example, the structural characteristics of certain stem-loop structures, such as tetraloop structures, attached to either end of a target gene sequence, render them initially processable by Class I, Class II, and/or Class III RNase III endoribonucleases, but not Class IV DICER nucleases. Cleavage of these stem-loop structures creates asymmetric, biologically active siRNA polynucleotides that are then recognizable by RISC complexes, and which provide greater target specificity than other double-stranded antisense molecules in the art.

In particular embodiments, precursor polynucletoides comprise a targeting region complementary to a target gene sequence, as well as two self-complementary regions that form stem-loop structures, one located at each end of the targeting region. In one embodiment, both self-complementary regions are recognized and cleaved by a RNase III endoribonuclease that is not a class IV DICER endoribonuclease. In another embodiment, one self-complementary region is recognized and cleaved by a RNase III endoribonuclease that is not a class IV DICER endoribonuclease, while the other self-complementary region is recognized and cleaved by a class IV DICER endoribonuclease. Thus, in certain embodiments, the resulting active asymmetric RNAi molecule is not produced until it is in the nucleus of the cell, thus reducing the possibility of undesired immune responses. In various embodiments, either type of self-complementary region may be at either end of the polynucleotide.

The invention is based, in part, upon the presence in the precursor polynucleotides of the invention of one or more self-complementary regions being capable of forming a stem-loop structure, attracting pre-processing endonucleases, and being recognized by dsRNA binding domains of recruited proteins. Furthermore, the compositions in this invention are unique in that they provide a rational method for triggering RNAi, but do so without the symmetrical dsRNA found in other siRNA and shRNA precursors.

The invention is also based on upon the recognition of the post-processed asymmetric polynucleotide structure guiding a protein complex using only one complete strand complementary to a gene or an mRNA. This “asymmetric” function results in a markedly more specific in inhibition of a target gene than that of dsRNA, while utilizing many of the same endogenous mechanisms. In contrast to the use of typical double-stranded antisense molecules, the “asymmetric” function of the instant polynucleotides, which still contain double stranded regions, reduces or eliminates the risk of unexpected or undesired targeting by the reverse strand.

Finally, the invention provides compositions that uniquely control the pathway leading to gene modulation. The precursors are step-processed and can be designed to either utilize or avoid endogenous complexes that lead to traditional gene silencing.

Given their effectiveness, the compositions of the present invention may be delivered to a cell or subject with an accompanying expectation of specificity predicted by the single strand complementary to the mRNA.

In accordance with the present invention, self-forming precursor polynucleotides, which comprise a nucleotide sequence with complementarity to an mRNA expressed from a target gene, as well as one or more self-complementary regions, are used to regulate gene expression. As used herein, a self-forming precursor polynucleotide means an isolated polynucleotide comprising a single-stranded region complementary to a region of a target mRNA or gene sequence, and one or more self-complementary regions located at both of the 5′ and 3′ ends of the polynucleotide, and which are capable of forming a double-stranded region, such as a stem-loop structure.

Self-forming precursor polynucleotides are interchangeably referred to herein as self-forming oligonucleotide precursors. Self-forming precursor polynucleotides of the present invention offer surprising advantages over polynucleotide inhibitors of the prior art, including antisense RNA and RNA interference molecules, including increased stability and increased effectiveness, such as by decreased off-target effects.

In certain embodiments, self-forming precursor polynucleotides comprise two or more regions of sequence complementary to a target gene, or target gene sequences. In particular embodiments, these regions are complementary to the same target genes or mRNAs, while in other embodiments, they are complementary to two or more different target genes or mRNAs. Accordingly, the present invention includes self-complementary polynucleotides comprising a series of sequences complementary to one or more target mRNAs or genes. In particular embodiments, these sequences are separated by regions of sequence that are non-complementary or semi-complementary to a target mRNA sequence and non-complementary to a self-complementary region. In other embodiments of self-forming precursor polynucleotides comprising multiple sequence complementary to target genes or mRNAs, the self-forming precursor polynucleotide comprises a self-complementary region at the 5′, 3′, or both ends of one or more regions of sequence complementary to a target gene. In a particular embodiment, a self-forming precursor polynucleotide comprises two or more regions of sequence complementary to one or more target genes, with self-complementary regions located at the 5′ and 3′ end of each region complementary to a target gene.

As used herein, the term “self-complementary” refers to a nucleotide sequence wherein a first region of the nucleotide sequence binds to a second region of the nucleotide sequence to form A-T(U) and G-C hybridization pairs. The two regions of the nucleotide sequence that bind to each other may be contiguous or may be separated by other nucleotides. The term “non-complementary” indicates that in a particular stretch of nucleotides, there are no nucleotides within that align with a target to form A-T(U) or G-C hybridizations. The term “semi-complementary” indicates that in a stretch of nucleotides, there is at least one nucleotide pair that aligns with a target to form an A-T(U) or G-C hybridizations, but there is not a sufficient number of complementary nucleotide pairs to support binding within the stretch of nucleotides under physiological conditions.

The term isolated refers to a material that is at least partially free from components that normally accompany the material in the material's native state. Isolation connotes a degree of separation from an original source or surroundings. Isolated, as used herein, e.g., related to DNA, refers to a polynucleotide that is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

In various embodiments, a self-forming precursor polynucleotide of the present invention comprises RNA, DNA, or peptide nucleic acids, or a combination of any or all of these types of molecules. In addition, a self-forming precursor polynucleotide may comprise modified nucleic acids, or derivatives or analogs of nucleic acids.

Examples of nucleic acid modifications include, but are not limited to, biotin labeling, fluorescent labeling, amino modifiers introducing a primary amine into the polynucleotide, phosphate groups, deoxyuridine, halogenated nucleosides, phosphorothioates, 2′-OMe RNA analogs, chimeric RNA analogs, wobble groups, and deoxyinosine.

The term “analog” as used herein refers to a molecule, compound, or composition that retains the same structure and/or function (e.g., binding to a target) as a polynucleotide herein. Examples of analogs include peptidomimetics, peptide nucleic acids, and small and large organic or inorganic compounds.

The term “derivative” or “variant” as used herein refers to a polynucleotide that differs from a naturally occurring polynucleotide (e.g., target gene sequence) by one or more nucleic acid deletions, additions, substitutions or side-chain modifications. In certain embodiments, variants have at least 70%, at least 80% at least 90%, at least 95%, or at least 99% sequence identity to a region of a target gene sequence. Thus, for example, in certain embodiments, a self-forming precursor oligonucleotide of the present invention comprises a region that is complementary to a variant of a target gene sequence.

In each case, self-forming precursor polynucleotides of the present invention comprise a sequence region that is complementary, and more preferably, completely complementary to one or more regions of a target gene or polynucleotide sequence (or a variant thereof). In certain embodiments, selection of a sequence region complementary to a target gene (or mRNA) is based upon analysis of the chosen target sequence and determination of secondary structure, Tₘ, binding energy, and relative stability. Such sequences may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17):3389-402) or Oligoengine Workstation 2.0.

In one embodiment, target sites are preferentially not located within the 5′ and 3′ untranslated regions (UTRs) or regions near the start codon (within approximately 75 bases), since proteins that bind regulatory regions may interfere with the binding of the polynucleotide. In addition, potential target sites may be compared to an appropriate genome database, such as BLASTN 2.0.5, available on the NCBI server at www.ncbi.nlm, and potential target sequences with significant homology to other coding sequences eliminated.

In another embodiment, the target sites are located within the 5′ or 3′ untranslated region (UTRs). In addition, the self-complementary of the self-forming precursor polynucleotide may be composed of a particular sequence found in the mRNA of the target.

In another embodiment, the loop region may designed to form a kissing-loop interaction with a determined loop region found in the 5′ or 3′ untranslated region (UTRs) of the target gene or a secondary target gene to that of the self-forming precursor polynucleotide.

The target gene or mRNA may be from an organism of any species, including, for example, plant, animal (e.g. mammalian), protozoan, viral, bacterial or fungal.

As noted above, the target gene sequence and the complementary region of the self-forming precursor polynucleotide may be complete complements of each other, or they may be less than completely complementary, i.e., partially complementary, as long as the strands hybridize to each other under physiological conditions.

Self-forming precursor polynucleotides of the present invention comprise a region complementary to a target mRNA or gene, as well as one or more self-complementary regions. In addition, they may optionally comprise one or more gap regions located between the region complementary to a target mRNA or gene and a self-complementary region.

Typically, the region complementary to a target mRNA or gene is 17 to 30 nucleotides in length, including integer values within these ranges, such as 19 or 21 nts. This region may be at least 16 nucleotides in length, at least 17 nucleotides in length, at least 20 nucleotides in length, at least 24 nucleotides in length, between 16 and 24 nucleotides in length, or between 17 and 24 nucleotides in length, including any integer value within these ranges.

The self-complementary region is typically between 14 and 54 nucleotides in length, at least 14 nucleotides in length, at least 16 nucleotides in length, or at least 20 nucleotides in length, including any integer value within any of these ranges (e.g., at least 15, 17, 18, 19, 21, 22, 23, 24, 24, 26, 27, 28, 20, 30, 31, 32, 33, 34, 35, 36, 37, 38, 39, 40, 41, 42, 43, 44, 45, 46, 47, 48, 49, 50, 51, 52, 53 nucleotides in length). In certain embodiments, the self-complementary region is located at the 5′ or 3′ end of the polynucleotide, and flanks the one or more target gene sequences. When the polynucleotide comprises two self-complementary regions, in certain embodiments, one is located at the 5′ end and one is located at the 3′ end.

In preferred embodiments, a self-complementary region is long enough to form a double-stranded structure. In one embodiment, a self-complementary region forms a stem-loop structure comprising a double-stranded region of self-complementary sequence and a loop of single-stranded sequence. Accordingly, in one embodiment, the primary sequence of a self-complementary region comprises two stretches of sequence complementary to each other separated by additional sequence that is not complementary or is semi-complementary. While less optimal, the additional sequence can be complementary in certain embodiments.

The additional sequence forms the loop of the stem-loop structure and, therefore, should be long enough to facilitate the folding necessary to allow the two complementary stretches to bind each other. In particular embodiments, the loop sequence comprises at least 2, at least 3, at least 4, at least 5, at least 6 bases, or 7, 8, 9, 10, 11, 12, 13, 14, 15, or 16 bases. In one embodiment, the loop sequence comprises 4 bases, also referred to herein as a tetraloop structure. The two stretches of sequence complementary to each other (within the self-complementary region; i.e., the stem regions) are typically of sufficient length to specifically hybridize to each other under physiological conditions. In certain embodiments, each stretch comprises 4 to 12 base-paired nucleotides; in other embodiments, each stretch comprises at least 4, at least 5, at least 6, at least 8, at least 10 nucleotides, at least 12 nucleotides, at least 14 nucleotides, at least 16 nucleotides, at least 18 nucleotides, at least 20 nucleotides, or any integer value within these ranges. In a particular embodiment, a self-complementary region comprises two stretches of at least 4 complementary nucleotides separated by a loop sequence of at least 4 nucleotides.

Furthermore, when a self-forming precursor polynucleotide comprises two or more self-complementary regions, such as a first self-complementary regions and a second self-complementary region, the two regions are not complementary to each other. Additionally, in certain embodiments, the self-complementary region is not complementary to the region of the self-forming precursor polynucleotide that is complementary to the target mRNA or gene.

In other embodiments, one or both self-complementary regions contain a sequence that is complementary to at least part of the target gene sequence. However, in these and related embodiments, when both self-complementary regions are complementary to at least part of the target gene sequence, there typically may remains a portion of the target gene sequence that is not complementary to either self-complementary region, such that when the self-forming polynucleotide has adopted its stem-loop structure, a portion of the target sequence present in the targeting region does not have a complementary sequence in either of the self-complementary regions. Thus, a portion of the target gene sequence remains single stranded. This single stranded region of the target gene sequence may consist of about 1, 2, 3, 4, 5, 6, 7, 8, 9, 10 or more contiguous nucleotides.

In particular embodiments, self-complementary regions possess thermodynamic parameters appropriate for binding of self-complementary regions, e.g., to form a stem-loop structure.

In one embodiment, self-complementary regions are dynamically calculated by use of RNA via free-energy analysis and then compared to the energy contained within the remaining “non self-complementary region” or loop region to ensure that the energy composition is adequate to form a desired structure, e.g., a stem-loop structure. In general, different nucleotide sequences of the mRNA targeting region are considered in determining the compositions of the stem-loops structures to ensure the formation of such. The free-energy analysis formula may again be altered to account for the type of nucleotide or pH of the environment in which it is used. Many different secondary structure prediction programs are available in the art, and each may be used according to the invention. Thermodynamic parameters for RNA and DNA bases are also publicly available in combination with target sequence selection algorithms, of which several are available in the art.

In one embodiment, the self-forming precursor polynucleotide comprises or consists of (a) a sequence comprising 17 to 30 nucleotides in length (including any integer value in-between), which is complementary to and capable of hybridizing under physiological conditions to at least a portion of an mRNA molecule, flanked optionally by (b) two gap regions comprising 1 to 4 nucleotide in length, and (c) two self-complementary sequences comprising 16 to 54 nucleotides in length (including any integer value in-between). In certain embodiments, each self-complementary sequence is capable of forming a stem-loop structure, one of which is located at the 5′ end and one of which is located at the 3′ end of the self-forming precursor polynucleotide.

In certain embodiments, the self-complementary region functions to inhibit or reduce degradation of the self-forming precursor oligonucleotide under physiological conditions, such as the conditions within a cell. Without wishing to be bound to a particular theory, it is believed that the structure adopted by a self-complementary region makes the polynucleotide more resistant to nuclease degradation than those lacking a self-complementary region. In addition, the presence of the structure adopted by the self-complementary regions is believed to facilitate cellular uptake and reduce undesired side effects. Accordingly, in various embodiments, a self-forming precursor polynucleotide has an increased in vivo half-life as compared to the same polynucleotide lacking self-complementary regions, as described herein. The half-life may be increased by at least 2-fold, at least 3-fold, at least 4-fold, at least 5-fold, or at least 10-fold, in various embodiments.

In certain embodiments, after entry into a target cell, the self-complementary regions function as a structure to recruit enzymatic cleavage of itself and/or bind to particular regions of proteins involved in the catalytic process of gene modulation. In certain embodiments, the proteins involved in enzymatic cleavage of the instant precursor polynucleotides include RNase III endoribonucleases, which specifically bind to and cleave double-stranded RNA (dsRNA).

RNase III endoribonucleases typically fall into one of four classes (see, e.g., Lamontagne et al. The Journal of Biological Chemistry. 279:2231-2241, 2004). Class I RNases III are largely found in bacteria and bacteriophage, and include all bacterial enzymes that possess both the classical nuclease domain and a dsRNA binding domain. Exemplary Class I RNase III endoribonucleases include rnc from E. coli.

Class II enzymes are typically distinguished from Class I enzymes by the presence of an N-terminal extension. Examples of Class II endoribonucleases include PacI from Saccharomyces pombe, and Rnt1p from S. cerevisiae.

Class III enzymes typically possess two nuclease domains and include both plant and vertebrate enzymes. Examples of Class III enzymes include Drosha proteins (see, e.g., Filippov, et al., Gene. 245:213-221, 2000). Drosha enzymes are typically responsible for initiating the processing of microRNA (miRNA), or short RNA molecules naturally expressed by the cell that regulate a wide variety of other genes by interacting with the RISC complex to induce cleavage of complementary mRNA. Drosha exists as part of a protein complex called the Microprocessor complex, which also contains the double-stranded RNA binding protein Pasha (also called DGCR8; see Denli et al., Nature 432:231-5, 2004), which is essential for Drosha activity and is capable of binding single-stranded fragments of the pri-miRNA that are required for proper processing (see Han et al., Cell 125:887-901, 2006). Both Drosha and Pasha are localized to the cell nucleus, where processing of pri-miRNA to pre-miRNA occurs. This latter molecule is then further processed by the RNase DICER into mature miRNAs in the cell cytoplasm.

Class IV RNase III endoribonucleases include the DICER and DICER-like family of enzymes, which are known to function in RNA interference (RNAi). DICER is an endoribonuclease in the RNase III family that cleaves double-stranded RNA (dsRNA) and pre-microRNA (miRNA) into short double-stranded RNA fragments (see Bernstein et al., Nature 409:363-6, 2001). These short double-stranded RNA fragments are often referred to as small interfering RNA (siRNA), which are typically about 20-25 nucleotides long, and usually contain a two-base overhang on the 3′ end. DICER enzymes contain dual RNase III domains/motifs and one PAZ domain (see Song et al., Nat. Struct. Biol. 10:1026-32, 2003, for the structure of PAZ domains), and the distance between these two regions of the molecule is determined by the length and angle of the connector helix and determines the length of the siRNAs it produces (see Macrae, et al., Science 311: 195-8, 2006). DICER catalyzes the first step in the RNA interference pathway, and initiates formation of the RISC, whose catalytic component argonaute is an endonuclease that is capable of degrading messenger RNA (mRNA) having a sequence that is complementary to that of the siRNA guide strand, or target gene sequence (see, Jaronczyk et al., Biochem J. 387:561-71, 2005).

As one example of a polynucleotide structure that is capable of recruiting a RNase III endoribonuclease that is not a DICER enzyme, the dsRNA substrates of Saccharomyces cerevisiae RNase III (Rnt1p) are capped by tetraloops with the consensus sequence (U/A)GNN, which act as the primary docking site for the RNase (see, e.g., Gaudin et al., J Mol. Biol. 363:322-31, 2006). The solution structures of two RNA hairpins capped by AGNN tetraloops, AGAA and AGUU, have been solved using NMR spectroscopy (see, e.g., Wu et al., EMBO J. 20:7240-9, 2001). These tetraloops have the same overall structure, in which the backbone turn occurs on the 3′ side of the syn G residue in the loop, with the first A and G in a 5′ stack and the last two residues in a 3′ stack. Also, a non-bridging phosphate oxygen and the universal G, which contributes to Rnt1p binding, are typically exposed. The compared biochemical and structural analysis of various tetraloop sequences exemplifies a family of RNA tetraloop structures with the consensus (U/A)GNN, and implicates this conserved structure as the primary determinant for specific recognition of Rnt1p substrates. Mammalian cells, including human cells, also comprise Rnt1 endoribonucleases (see, e.g., Wu et al., J. Biol. Chem., 275:36957-36965, 2000; MacRae et al., Curr Opin Struct Biol. 17:1-8, 2007; Mathieu et al., Molecular Biology of the Cell 15:3015-3030, 2004), which may be recruited to tetraloop containing stem-loop structures, or to other stem-loop structures, as described herein.

Thus, in certain embodiments, the loop structure contained within a self-complementary region may be of a certain 4-nucleotide tetraloop structure (e.g., NGNN, AAGU, (U/A)GNN, AGAA and AGUU) to promote the initial cleavage of that self-complementary region by certain Class I, Class II, and Class III RNase III endoribonucleases, such as Rnt1, but not by DICER-like enzymes.

Alternatively, or in combination with a tetraloop structure, certain self-complementary regions may comprise a proximal box nucleotide sequence and/or a distal box nucleotide sequence, the terms proximal and distal being in relation to the gene target sequence (see FIGS. III(H) and (I)). In certain embodiments, the proximal and/or distal boxes represent a double-stranded RNA binding domain, and facilitate the interaction of the stem-loop structure with the Class I, Class II, or Class III RNase III endoribonuclease that is not a DICER enzyme. In certain embodiments, the proximal box sequence is GAAA, or any other 4 nucleotide sequence that typically excludes a guanine at position 2. In certain embodiments, the distal box sequence is a two nucleotide segment, which may include 5′-AG-3′ duplexed with UU.

Thus, certain embodiments may comprise at least one self-complementary region that comprises a tetraloop structure, a proximal box, and/or a distal box sequence. Other embodiments may comprise at least one self-complementary region that has a stem-loop structure as described elsewhere herein (e.g., 2-14 nucleotides), which may or may not form a tetraloop structure, and which comprises a proximal box sequence and/or a distal box sequence.

Typically, these self-complementary regions are not intially processable by Class IV DICER endoribonucleases, at least until one of the regions has been first processed by a Class I, Class II, or Class III endoribonuclease. For instance, in these and other embodiments, the self-complementary region can be cleaved by Rnt1 or Pad at 11/13 or 14/16 nucleotides into the duplex region leaving a 2 nucleotide 3′ end.

In certain embodiments, the self-complementary region that has been first enzymatically cleaved by a Class I, Class II, or Class RNase III endoribonucleases that is not a Class IV DICER enzyme, will afterwards be capable of loading onto, or interacting with, the protein region of DICER, known as PAZ (PIWI, Argonaute, Zwille), and/or RISC complexes. In these and other embodiments, the post-cleavage, self-complementary region may remain in helical form of 5-14 base pairs to be recognized by the dsRNA Binding Domains (dsRBD) of DICER and/or RISC complexes.

In certain embodiments, a self-forming precursor polynucleotide of the invention comprises at least one self-complementary region that contains a stem-loop structure, such as a tetraloop structure, and/or a combination of proxima/distal box sequences, and which is processable by a Class I, Class II, or Class III RNase III endoribonuclease, such as Rnt1, Pac1, and/or mc. In certain embodiments, a self-forming precursor polynucleotide comprises two self-complementary regions, one at each end of the one or more target gene sequences, each of which stem-loop structure, such as a tetraloop structure, and/or a combination of proxima/distal box sequences, and which is processable by a Class I, Class II, or Class III RNase III endoribonuclease that is not a Class IV DICER enzyme. In these and related embodiments, both ends of the precursor polynucleotide are first processable by a Class I, Class II, and/or Class III RNase III endoribonuclease that is not a Class IV DICER enzyme, and after that processing (see, e.g., FIG. VII), are then typically capable of interacting with the dsRBD of DICER and/or RISC complexes.

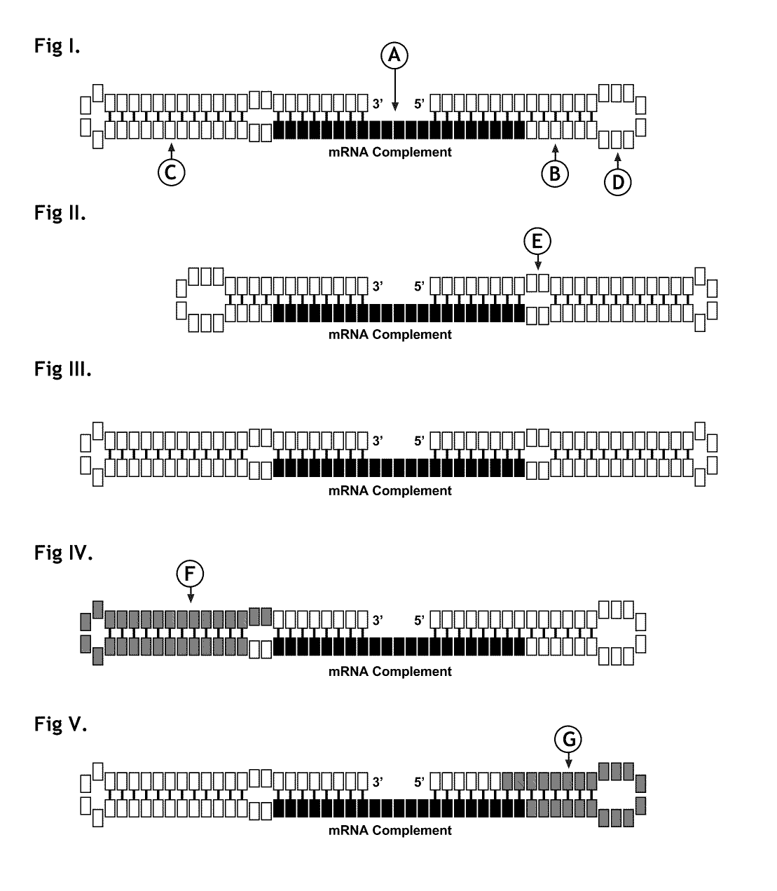

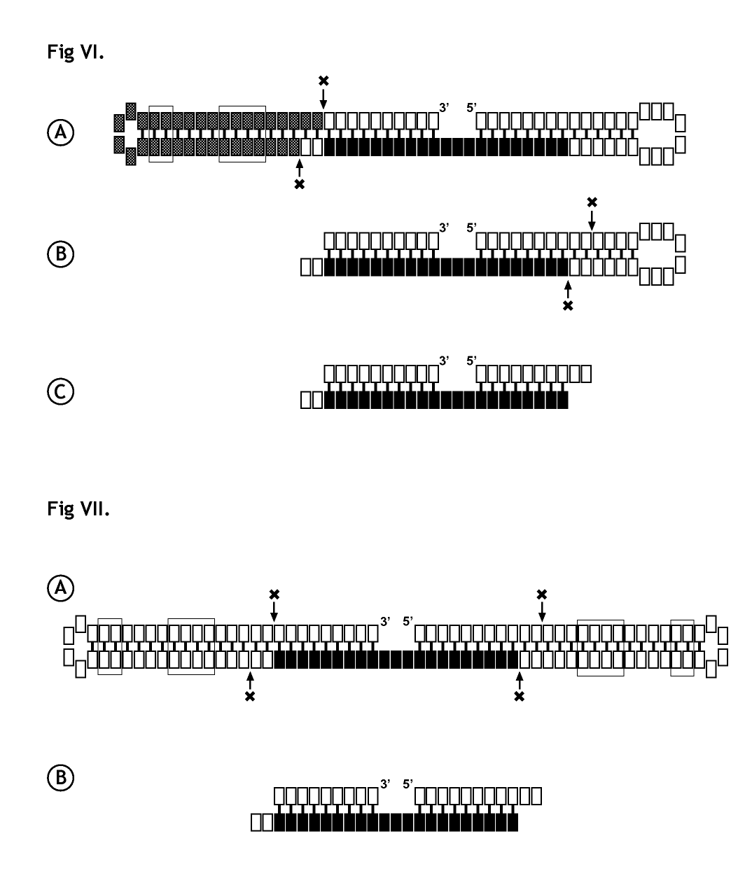

In certain embodiments, one of the self-complementary regions may form a loop structure that is greater than 4 nucleotides (e.g., 5-16, 5-14, 6, 7, 8, 9, 10, 11, 12, 13, 14, 15, or 16 nucleotides) (see, e.g., FIG. 1(D), which can prevent the cleavage of that self-complementary region by certain RNase III endoribonucleases, such as Rnt1 from yeast, i.e., the loop structure renders that region resistant to cleavage by an RNase III enzyme such as Rnt1. In these and related embodiments, the self-complementary region typically also comprises a double-stranded region of about 5-16 or 5-14 base pairs (see, e.g., FIG. I(B), often in helical form, which can be recognized by the dsRNA Binding Domains (dsRBD) of DICER and/or RISC complexes.

Therefore, in certain embodiments, a self-forming precursor polynucleotide may comprise two self-complementary regions, one at each end of the one or more target gene sequences, the first of which contains or forms a stem-loop structure, such as tetraloop structure, and which may contain a proximal and distal box, wherein the region is processable by a Class I RNase III endoribonuclease such as Rnt1, and the second of which contains or forms a loop structure that is greater than 4 nucleotides (e.g., about 5-14 nucleotides), which may be resistant to cleavage by a Class I RNase III enzyme such as Rnt1, but which can be recognized and cleaved by DICER after the cleavage of the first self-complementary region. In these and related embodiments (see, e.g., FIG. I), without being bound by any one theory, the first self-complementary region may be cleaved by an RNase III such as Rnt1, which allows the remaining polynucleotide structure (see, e.g., FIG. VI(B)), having the loop structure of about 5-14 nucleotides, to be recognized and processed by DICER enzymes, thereby forming an asymmetric, biologically active siRNA polynucleotide that is capable of interacting with and activating RISC complex activity towards the complement of the gene target sequence.

In preferred embodiments, self-forming precursor polynucleotides of the present invention bind to and reduce expression of a target mRNA. A target gene may be a known gene target, or, alternatively, a target gene may be not known, i.e., a random sequence may be used. In certain embodiments, target mRNA levels are reduced at least 10%, at least 20%, at least 30%, at least 40%, at least 50%, at least 60%, at least 70%, at least 75%, at least 80%, at least 90%, or at least 95%.

In one embodiment of the invention, the level of inhibition of target gene expression (i.e., mRNA expression) is at least 90%, at least 95%, at least 98%, at least 99% or is almost 100%, and hence the cell or organism will in effect have the phenotype equivalent to a so-called “knock out” of a gene. However, in some embodiments, it may be preferred to achieve only partial inhibition so that the phenotype is equivalent to a so-called “knockdown” of the gene. This method of knocking down gene expression can be used therapeutically or for research (e.g., to generate models of disease states, to examine the function of a gene, to assess whether an agent acts on a gene, to validate targets for drug discovery).

The invention further provides arrays of self-forming precursor polynucleotides of the invention, including microarrays. Microarrays are miniaturized devices typically with dimensions in the micrometer to millimeter range for performing chemical and biochemical reactions and are particularly suited for embodiments of the invention. Arrays may be constructed via microelectronic and/or microfabrication using essentially any and all techniques known and available in the semiconductor industry and/or in the biochemistry industry, provided only that such techniques are amenable to and compatible with the deposition and/or screening of polynucleotide sequences.

Microarrays of the invention are particularly desirable for high throughput analysis of multiple self-forming precursor polynucleotides. A microarray typically is constructed with discrete region or spots that comprise self-forming precursor polynucleotides of the present invention, each spot comprising one or more self-forming precursor polynucleotide, preferably at positionally addressable locations on the array surface. Arrays of the invention may be prepared by any method available in the art. For example, the light-directed chemical synthesis process developed by Affymetrix (see, U.S. Pat. Nos. 5,445,934 and 5,856,174) may be used to synthesize biomolecules on chip surfaces by combining solid-phase photochemical synthesis with photolithographic fabrication techniques. The chemical deposition approach developed by Incyte Pharmaceutical uses pre-synthesized cDNA probes for directed deposition onto chip surfaces (see, e.g., U.S. Pat. No. 5,874,554).

In certain embodiments, a self-forming precursor polynucleotide of the present invention is synthesized using techniques widely available in the art. In other embodiments, it is expressed in vitro or in vivo using appropriate and widely known techniques. Accordingly, in certain embodiments, the present invention includes in vitro and in vivo expression vectors comprising the sequence of a self-forming precursor polynucleotide of the present invention. Methods well known to those skilled in the art may be used to construct expression vectors containing sequences encoding a self-forming precursor polynucleotide, as well as appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

Expression vectors typically include regulatory sequences, which regulate expression of the self-forming precursor polynucleotide. Regulatory sequences present in an expression vector include those non-translated regions of the vector, e.g., enhancers, promoters, 5′ and 3′ untranslated regions, which interact with host cellular proteins to carry out transcription and translation. Such elements may vary in their strength and specificity. Depending on the vector system and cell utilized, any number of suitable transcription and translation elements, including constitutive and inducible promoters, may be used. In addition, tissue- or -cell specific promoters may also be used.

For expression in mammalian cells, promoters from mammalian genes or from mammalian viruses are generally preferred. In addition, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

In certain embodiments, the invention provides for the conditional expression of a self-forming precursor polynucleotide. A variety of conditional expression systems are known and available in the art for use in both cells and animals, and the invention contemplates the use of any such conditional expression system to regulate the expression or activity of a self-forming precursor polynucleotide. In one embodiment of the invention, for example, inducible expression is achieved using the REV-TET system. Components of this system and methods of using the system to control the expression of a gene are well documented in the literature, and vectors expressing the tetracycline-controlled transactivator (tTA) or the reverse tTA (rtTA) are commercially available (e.g., pTet-Off, pTet-On and ptTA-2/3/4 vectors, Clontech, Palo Alto, Calif.). Such systems are described, for example, in U.S. Pat. No. 5,650,298,U.S. Pat. No. 6,271,348, U.S. Pat. No. 5,922,927, and related patents, which are incorporated by reference in their entirety. In one particular embodiment, self-forming precursor polynucleotides are expressed using a vector system comprising a pSUPER vector backbone and additional sequences corresponding to the self-forming precursor polynucleotide to be expressed. The pSUPER vectors system has been shown useful in expressing siRNA reagents and downregulating gene expression (Brummelkamp, T. T. et al., Science 296:550 (2002) and Brummelkamp, T. R. et al., Cancer Cell, published online Aug. 22, 2002). PSUPER vectors are commercially available from OligoEngine, Seattle, Wash.

### Methods of Regulating Gene Expression

Self-forming precursor polynucleotides of the invention may be used for a variety of purposes, all generally related to their ability to inhibit or reduce expression of a target gene. Accordingly, the invention provides methods of reducing expression of one or more target genes comprising introducing a self-forming precursor polynucleotide of the invention into a cell that contains a target gene or a homolog, variant or ortholog thereof. In addition, self-forming precursor polynucleotides may be used to reduce expression indirectly. For example, a self-forming precursor polynucleotide may be used to reduce expression of a transactivator that drives expression of a second gene, thereby reducing expression of the second gene. Similarly, a self-forming precursor polynucleotide may be used to increase expression indirectly. For example, a self-forming precursor polynucleotide may be used to reduce expression of a transcriptional repressor that inhibits expression of a second gene, thereby increasing expression of the second gene.

In various embodiments, a target gene is a gene derived from the cell into which a self-forming precursor polynucleotide is to be introduced, an endogenous gene, an exogenous gene, a transgene, or a gene of a pathogen that is present in the cell after transfection thereof. Depending on the particular target gene and the amount of the self-forming precursor polynucleotide delivered into the cell, the method of this invention may cause partial or complete inhibition of the expression of the target gene. The cell containing the target gene may be derived from or contained in any organism (e.g., plant, animal, protozoan, virus, bacterium, or fungus).

Inhibition of the expression of the target gene can be verified by means including, but not limited to, observing or detecting an absence or observable decrease in the level of protein encoded by a target gene, and/or mRNA product from a target gene, and/or a phenotype associated with expression of the gene, using techniques known to a person skilled in the field of the present invention.

Examples of cell characteristics that may be examined to determine the effect caused by introduction of a self-forming precursor polynucleotide of the invention include, cell growth, apoptosis, cell cycle characteristics, cellular differentiation, and morphology.

A self-forming precursor polynucleotide may be directly introduced to the cell (i.e., intracellularly), or introduced extracellularly into a cavity, interstitial space, into the circulation of an organism, introduced orally, by bathing an organism in a solution containing the self-forming precursor polynucleotide, or by some other means sufficient to deliver the self-forming precursor polynucleotide into the cell.

In addition, a vector engineered to express a self-forming precursor polynucleotide may be introduced into a cell, wherein the vector expresses the self-forming precursor polynucleotide, thereby introducing it into the cell. Methods of transferring an expression vector into a cell are widely known and available in the art, including, e.g., transfection, lipofection, scrape-loading, electroporation, microinjection, infection, gene gun, and retrotransposition. Generally, a suitable method of introducing a vector into a cell is readily determined by one of skill in the art based upon the type of vector and the type of cell, and teachings widely available in the art. Infective agents may be introduced by a variety of means readily available in the art, including, e.g., nasal inhalation.

Methods of inhibiting gene expression using self-forming precursor oligonucleotides of the invention may be combined with other knockdown and knockout methods, e.g., gene targeting, antisense RNA, ribozymes, double-stranded RNA (e.g., shRNA and siRNA) to further reduce expression of a target gene.

In different embodiments, target cells of the invention are primary cells, cell lines, immortalized cells, or transformed cells. A target cell may be a somatic cell or a germ cell. The target cell may be a non-dividing cell, such as a neuron, or it may be capable of proliferating in vitro in suitable cell culture conditions. Target cells may be normal cells, or they may be diseased cells, including those containing a known genetic mutation. Eukaryotic target cells of the invention include mammalian cells, such as, for example, a human cell, a murine cell, a rodent cell, and a primate cell. In one embodiment, a target cell of the invention is a stem cell, which includes, for example, an embryonic stem cell, such as a murine embryonic stem cell.

The self-forming precursor polynucleotides and methods of the present invention may be used to treat any of a wide variety of diseases or disorders, including, but not limited to, inflammatory diseases, cardiovascular diseases, nervous system diseases, tumors, demyelinating diseases, digestive system diseases, endocrine system diseases, reproductive system diseases, hemic and lymphatic diseases, immunological diseases, mental disorders, muscoloskeletal diseases, neurological diseases, neuromuscular diseases, metabolic diseases, sexually transmitted diseases, skin and connective tissue diseases, urological diseases, and infections.

In certain embodiments, the methods are practiced on an animal, in particular embodiments, a mammal, and in certain embodiments, a human.

Accordingly, in one embodiment, the present invention includes methods of using a self-forming precursor oligonucleotide for the treatment or prevention of a disease associated with gene deregulation, overexpression, or mutation. For example, a self-forming precursor polynucleotide may be introduced into a cancerous cell or tumor and thereby inhibit expression of a gene required for or associated with maintenance of the carcinogenic/tumorigenic phenotype. To prevent a disease or other pathology, a target gene may be selected that is, e.g., required for initiation or maintenance of a disease/pathology. Treatment may include amelioration of any symptom associated with the disease or clinical indication associated with the pathology.

In addition, self-forming precursor polynucleotides of the present invention are used to treat diseases or disorders associated with gene mutation. In one embodiment, a self-forming precursor polynucleotide is used to modulate expression of a mutated gene or allele. In such embodiments, the mutated gene is the target of the self-forming precursor polynucleotide, which will comprise a region complementary to a region of the mutated gene. This region may include the mutation, but it is not required, as another region of the gene may also be targeted, resulting in decreased expression of the mutant gene or mRNA. In certain embodiments, this region comprises the mutation, and, in related embodiments, the resulting self-forming precursor oligonucleotides specifically inhibits expression of the mutant mRNA or gene but not the wild type mRNA or gene. Such a self-forming precursor polynucleotide is particularly useful in situations, e.g., where one allele is mutated but another is not. However, in other embodiments, this sequence would not necessarily comprise the mutation and may, therefore, comprise only wild-type sequence. Such a self-forming precursor polynucleotide is particularly useful in situations, e.g., where all alleles are mutated. A variety of diseases and disorders are known in the art to be associated with or caused by gene mutation, and the invention encompasses the treatment of any such disease or disorder with a self-forming precursor polynucleotide. For example, in one embodiment, cancer is treated using a self-forming precursor polynucleotide that targets a p53 gene or allele. In certain embodiment, the p53 gene or allele is a mutant p53 gene or allele.

In certain embodiments, a gene of a pathogen is targeted for inhibition. For example, the gene could cause immunosuppression of the host directly or be essential for replication of the pathogen, transmission of the pathogen, or maintenance of the infection. In addition, the target gene may be a pathogen gene or host gene responsible for entry of a pathogen into its host, drug metabolism by the pathogen or host, replication or integration of the pathogen's genome, establishment or spread of an infection in the host, or assembly of the next generation of pathogen. Methods of prophylaxis (i.e., prevention or decreased risk of infection), as well as reduction in the frequency or severity of symptoms associated with infection, are included in the present invention. For example, cells at risk for infection by a pathogen or already infected cells, particularly human immunodeficiency virus (HIV) infections, may be targeted for treatment by introduction of a self-forming precursor polynucleotide according to the invention.

In other specific embodiments, the present invention is used for the treatment or development of treatments for cancers of any type. Examples of tumors that can be treated using the methods described herein include, but are not limited to, neuroblastomas, myelomas, prostate cancers, small cell lung cancer, colon cancer, ovarian cancer, non-small cell lung cancer, brain tumors, breast cancer, leukemias, lymphomas, and others.

The self-forming precursor polynucleotides and expression vectors (including viral vectors and viruses) may be introduced into cells in vitro or ex vivo and then subsequently placed into an animal to affect therapy, or they may be directly introduced to a patient by in vivo administration. Thus, the invention provides methods of gene therapy, in certain embodiments. Compositions of the invention may be administered to a patient in any of a number of ways, including parenteral, intravenous, systemic, local, oral, intratumoral, intramuscular, subcutaneous, intraperitoneal, inhalation, or any such method of delivery. In one embodiment, the compositions are administered parenterally, i.e., intraarticularly, intravenously, intraperitoneally, subcutaneously, or intramuscularly. In a specific embodiment, the liposomal compositions are administered by intravenous infusion or intraperitoneally by a bolus injection.

Compositions of the invention may be formulated as pharmaceutical compositions suitable for delivery to a subject. The pharmaceutical compositions of the invention will often further comprise one or more buffers (e.g., neutral buffered saline or phosphate buffered saline), carbohydrates (e.g., glucose, mannose, sucrose, dextrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The amount of self-forming precursor oligonucleotides administered to a patient can be readily determined by a physician based upon a variety of factors, including, e.g., the disease and the level of self-forming precursor oligonucleotides expressed from the vector being used (in cases where a vector is administered). The amount administered per dose is typically selected to be above the minimal therapeutic dose but below a toxic dose. The choice of amount per dose will depend on a number of factors, such as the medical history of the patient, the use of other therapies, and the nature of the disease. In addition, the amount administered may be adjusted throughout treatment, depending on the patient's response to treatment and the presence or severity of any treatment-associated side effects.

The invention further includes a method of identifying gene function in an organism comprising the use of a self-forming precursor polynucleotide to inhibit the activity of a target gene of previously unknown function. Instead of the time consuming and laborious isolation of mutants by traditional genetic screening, functional genomics envisions determining the function of uncharacterized genes by employing the invention to reduce the amount and/or alter the timing of target gene activity. The invention may be used in determining potential targets for pharmaceutics, understanding normal and pathological events associated with development, determining signaling pathways responsible for postnatal development/aging, and the like. The increasing speed of acquiring nucleotide sequence information from genomic and expressed gene sources, including total sequences for the yeast, D. melanogaster, and C. elegans genomes, can be coupled with the invention to determine gene function in an organism (e.g., nematode). The preference of different organisms to use particular codons, searching sequence databases for related gene products, correlating the linkage map of genetic traits with the physical map from which the nucleotide sequences are derived, and artificial intelligence methods may be used to define putative open reading frames from the nucleotide sequences acquired in such sequencing projects.

In one embodiment, a self-forming precursor oligonucleotide is used to inhibit gene expression based upon a partial sequence available from an expressed sequence tag (EST), e.g., in order to determine the gene's function or biological activity. Functional alterations in growth, development, metabolism, disease resistance, or other biological processes would be indicative of the normal role of the EST's gene product.

The ease with which a self-forming precursor polynucleotide can be introduced into an intact cell/organism containing the target gene allows the present invention to be used in high throughput screening (HTS). For example, solutions containing self-forming precursor polynucleotide that are capable of inhibiting different expressed genes can be placed into individual wells positioned on a microtiter plate as an ordered array, and intact cells/organisms in each well can be assayed for any changes or modifications in behavior or development due to inhibition of target gene activity. The function of the target gene can be assayed from the effects it has on the cell/organism when gene activity is inhibited. In one embodiment, self-forming precursor polynucleotides of the invention are used for chemocogenomic screening, i.e., testing compounds for their ability to reverse a disease modeled by the reduction of gene expression using a self-forming precursor polynucleotide of the invention.

If a characteristic of an organism is determined to be genetically linked to a polymorphism through RFLP or QTL analysis, the present invention can be used to gain insight regarding whether that genetic polymorphism might be directly responsible for the characteristic. For example, a fragment defining the genetic polymorphism or sequences in the vicinity of such a genetic polymorphism can be amplified to produce an RNA, a self-forming precursor polynucleotide can be introduced to the organism, and whether an alteration in the characteristic is correlated with inhibition can be determined.

The present invention is also useful in allowing the inhibition of essential genes. Such genes may be required for cell or organism viability at only particular stages of development or cellular compartments. The functional equivalent of conditional mutations may be produced by inhibiting activity of the target gene when or where it is not required for viability. The invention allows addition of a self-forming precursor polynucleotide at specific times of development and locations in the organism without introducing permanent mutations into the target genome. Similarly, the invention contemplates the use of inducible or conditional vectors that express a self-forming precursor polynucleotide only when desired.

The present invention also relates to a method of validating whether a gene product is a target for drug discovery or development. A self-forming precursor polynucleotide that targets the mRNA that corresponds to the gene for degradation is introduced into a cell or organism. The cell or organism is maintained under conditions in which degradation of the mRNA occurs, resulting in decreased expression of the gene. Whether decreased expression of the gene has an effect on the cell or organism is determined. If decreased expression of the gene has an effect, then the gene product is a target for drug discovery or development.

### Methods of Designing and Producing Self-forming Precursor Polynucleotides

The self-forming precursor polynucleotides of the present invention comprise a novel and unique set of functional sequences, arranged in a manner so as to adopt a secondary structure containing one or more double-stranded regions (typically a stem-loop structure), which imparts the advantages of the self-forming precursor polynucleotides. Accordingly, in certain embodiments, the present invention includes methods of designing self-forming precursor polynucleotide of the present invention. Such methods typically involve appropriate selection of the various sequence components of the self-forming precursor polynucleotide.

In one embodiment, the basic design of self-forming precursor polynucleotides is as follows:

### Design Motif:

(stemA)(loopA)(stemB)(target)(stemC)(loopB)(stemD)

Accordingly, in a related embodiment, a self-forming precursor polynucleotide is designed as follows:

a. Start with target nucleotide sequence. The length and composition dictates the length and sequence composition of all stem and loop regions.
b. Stem A & D may need specific nucleotides for enzyme compatibility.
c. Build candidate Stem A & B with (4-24) nucleotides that have melting temperature dominant to equal length region of target. Stem strands have A-T, G-C complimentarity to each other. Length and composition depend upon which endoribonuclease is chosen for pre-processing of the stem-loop structure.
d. Build candidate Stem C & D with (4-24) nucleotides that have melting temperature dominant to equal length region of target. Stem strands have A-T, G-C complimentarity to each other, but no complimentarity to Stem A & B. Length and composition depend upon which endoribonuclease is chosen for pre-processing of the stem-loop structure.
e. Build loop candidates with (4-12) A-T rich nucleotides into loop A & B. Length and composition depend upon which endoribonuclease is chosen for pre-processing of the stem-loop structure. Tetraloops as described are suggested for longer stems processed by Rnt1 or Pad RNase III endoribonucleases as drawn in (FIG. A.). Larger loops are suggested for preventing Rnt1 or Pad processing and placed onto shorter stems as drawn in (FIG. C, FIG. D.).
f. Form a contiguous sequence for each motif candidate.
g. Fold candidate sequence using software with desired parameters.
h. From output, locate structures with single stranded target regions which are flanked at either one or both ends with a desired stem/loop structure.

In one embodiment, a method of designing a polynucleotide sequence comprising one or more self-complementary regions for the regulation of expression of a target gene (i.e., a self-forming precursor polynucleotide), includes: (a) selecting a first sequence 17 to 30 nucleotides in length and complementary to a target gene; and (b) selecting one or more additional sequences 12 to 54 nucleotides in length, which comprises self-complementary regions and which are non-complementary to the first sequence.

These methods, in certain embodiments, include determining or predicting the secondary structure adopted by the sequences selected in step (b), e.g., in order to determine that they are capable of adopting a stem-loop structure.

Similarly, these methods can include a verification step, which comprises testing the designed polynucleotide sequence for its ability to inhibit expression of a target gene, e.g., in an in vivo or in vitro test system.

The invention further contemplates the use of a computer program to select sequences of a self-forming precursor polynucleotide, based upon the complementarity characteristics described herein. The invention, thus, provides computer software programs, and computer readable media comprising said software programs, to be used to select self-forming precursor polynucleotide sequences, as well as computers containing one of the programs of the present invention.

In certain embodiments, a user provides a computer with information regarding the sequence, location or name of a target gene. The computer uses this input in a program of the present invention to identify one or more appropriate regions of the target gene to target, and outputs or provides complementary sequences to use in the self-forming precursor polynucleotide of the invention. The computer program then uses this sequence information to select sequences of the one or more self-complementary regions of the self-forming precursor polynucleotide. Typically, the program will select a sequence that is not complementary to a genomic sequence, including the target gene, or the region of the self-forming precursor polynucleotide that is complementary to the target mRNA. Furthermore, the program will select sequences of self-complementary regions that are not complementary to each other. When desired, the program also provides sequences of gap regions. Upon selection of appropriate sequences, the computer program outputs or provides this information to the user.

The programs of the present invention may further use input regarding the genomic sequence of the organism containing the target gene, e.g., public or private databases, as well as additional programs that predict secondary structure and/or hybridization characteristics of particular sequences, in order to ensure that the self-forming precursor polynucleotide adopts the correct secondary structure and does not hybridize to non-target genes.

The present invention is based, in part, upon the surprising discovery that self-forming precursor polynucleotides, as described herein, are extremely effective in reducing target gene expression. The self-forming precursor polynucleotides offer significant advantages over previously described antisense RNAs, including increased stability or resistance to nucleases, and increased effectiveness. Furthermore, the self-forming precursor polynucleotides of the invention offer additional advantages over traditional dsRNA molecules used for siRNA, since the use of self-forming precursor polynucleotides substantially eliminates the off-target suppression associated with dsRNA molecules. Lastly, the processed pre-cursor of the present invention is not recognized by proteins that act as siRNA antagonists.

The practice of the present invention will employ a variety of conventional techniques of cell biology, molecular biology, microbiology, and recombinant DNA, which are within the skill of the art. Such techniques are fully described in the literature. See, for example, Molecular Cloning: A Laboratory Manual, 2nd Ed., ed. by Sambrook, Fritsch, and Maniatis (Cold Spring Harbor Laboratory Press, 1989); and DNA Cloning, Volumes I and II (D. N. Glover ed. 1985). Each of the references described herein are incorporated by reference in their entireties.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheetare incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### EXAMPLES

### Example 1

### Gene Modulation by an Asymmetric Si RNA Expression Vector System in Human Cells

This Example demonstrates the design and testing of an expression vector that expresses an exemplary self-forming precursor polynucleotide of the invention. As compared to typical double-stranded siRNA molecules or shRNA, these polynucleotides to have increased stability and be more effective in specifically reducing target gene expression, without causing significant or measurable modulation of non-targeted genes.

I. Design Parts: Various Polynucletoides of the Present Invention Comprise the following features:
1. Sense Sequence of Target Gene Sequence n19-n25 Listed in Sense Orientation.

5′-NNNNNNNNNNNNNNNNNNNNNN-3′

### 2. Stem-Loop 5′, SL5:

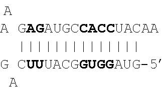

### 3. Stem-Loop 3′, SL3:

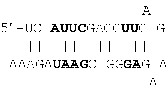

4. shRNA Stem-Loop

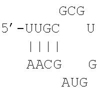

### II. Assemble Asymmetric RNAi Molecules (A-RNAi)

1. Design new target site or convert existing siRNA or shRNA into the Asymmetric version. This can be done manually, or one can use available tools such as those provided by Oligoengine, Inc. (Madison, Wis.) for rational Asymmetric RNAi designs.
A. Motif for Asymmetric siRNA (5′->3′): A-siRNA
(terminus of target sense)+(SL3)+(target site Reverse Comp.)+(SL5)+(leader of target sense)
B. Motif for Asymmetric shRNA (5′->3′): A-shRNA
(terminus of target sense)+(shRNA SL)+(target site Reverse Comp.)+(SL5)+(leader of target sense)
2. Append the A-RNAi motif DNA with the cloning sites, if needed. BgIII/HindIII used in this example

A. A-siRNA(Rnt1->RISC).
5′-GATCCCC(A-siRNA sequence)TTTTTA
5′-AGCTTAAAAA(A-siRNA RC sequence)GGG

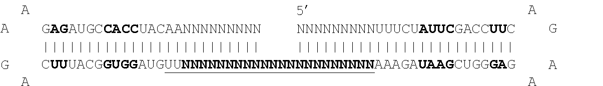

B. A-shRNA (Rnt1->Dicer->RISC)
5′-GATCCCC(A-shRNA sequence)TTTTTA
5′-AGCTTAAAAA(A-shRNA RC sequence)GGG

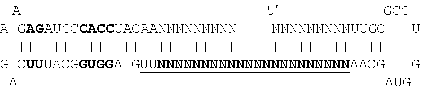

Asymmetric polynucleotides of the present invention may be compared to traditional RNAi molecules, including shRNA and siRNA molecules, as well as self-protected siRNA molecules, as described in U.S. patent application Ser. No. 11/813,190.

Positive Control shRNA:

A_Pos,
GATCCCCGCAAGCTGACCCTGAAGTTCTTCAAGAGAGAACTTCAGGGT
CAGCTTGCTTTTTA
A_Pos,
AGCTTAAAAAGCAAGCTGACCCTGAAGTTCTCTCTTGAAGAACTTCAG
GGTCAGCTTGCGGG

Negative Control shRNA:

A_Neg,
GATCCCCGCGCGCTTTGTAGGATTCGTTCAAGAGACGAATCCTACAAA
GCGCGCTTTTTA
A_Neg,
AGCTTAAAAAGCGCGCTTTGTAGGATTCGTCTCTTGAACGAATCCTAC
AAAGCGCGCGGG

III. Clone constructs using the pSUPER vector
1. Anneal the forward and reverse strands of the oligos that contain the Asymmeteric RNAi-expressing sequence targeting your gene of interest.
2. Linearize the pSUPER vector with BgIII and XhoI OR HindIII
3. Clone the annealed oligos into the vector
4. Transform the vector in bacteria
5. Transfect pSUPER vector into mammalian cells
6. Monitor EGFP fluorescence (for “+GFP” versions only)
7. Select with puromycin or neomycin to establish a stable cell line for siRNA expression (.neo or .puro versions)
8. Assay the effects on protein expression and/or mRNA levels

### IV. Example Assessment Protocol

Plate 15 k/well cells the day before transfection.

Co-transfect 10 ng of EmGFP, 60 ng of the pSUPER construct and Beta Lactamase reporter plasmid into cells in triplicate. The addition of Beta Lactamase is to transfect the same total amount of plasmids to ensure similar transfection efficiency across the samples. Change media 6 hours post-transfection with full growth medium containing serum.

Measure the fluorescence by flow cytometry 48-72 hours post-transfection. For each sample, about 2000 events are to be measured. Analyze data to assess EmGFP Knockdown. The remaining total fluorescence of each sample is normalized to the negative control.

The asymmetric polynucleotide of the present invention is predicted to reduce target gene expression as well as the other RNAi molecules tested, and have reduced levels of off-target suppression.

### Example 2

### Design and Production of Asymmetric Precursor Anti-GFP Polynucleotide

EGFP Sequence Obtained from pIRES-EGFP Vector Sequence

GFP DNA:
ATGGTGAGCAAGGGCGAGGAGCTGTTCACCGGGGTGGTGCCCATCCTG
GTCGAGCTGGACGGCGACGTAAACGGCCACAAGTTCAGCGTGTCCGGC
GAGGGCGAGGGCGATGCCACCTACGGCAAGCTGACCCTGAAGTTCATC
TGCACCACCGGCAAGCTGCCCGTGCCCTGGCCCACCCTCGTGACCACC
CTGACCTACGGCGTGCAGTGCTTCAGCCGCTACCCCGACCACATGAAG
CAGCACGACTTCTTCAAGTCCGCCATGCCCGAAGGCTACGTCCAGGAG
CGCACCATCTTCTTCAAGGACGACGGCAACTACAAGACCCGCGCCGAG
GTGAAGTTCGAGGGCGACACCCTGGTGAACCGCATCGAGCTGAAGGGC
ATCGACTTCAAGGAGGACGGCAACATCCTGGGGCACAAGCTGGAGTAC
AACTACAACAGCCACAACGTCTATATCATGGCCGACAAGCAGAAGAAC
GGCATCAAGGTGAACTTCAAGATCCGCCACAACATCGAGGACGGCAGC
GTGCAGCTCGCCGACCACTACCAGCAGAACACCCCCATCGGCGACGGC
CCCGTGCTGCTGCCCGACAACCACTACCTGAGCACCCAGTCCGCCCTG
AGCAAAGACCCCAACGAGAAGCGCGATCACATGGTCCTGCTGGAGTTC
GTGACCGCCGCCGGGATCACTCTCGGCATGGACGAGCTGTACAAGTAAA
GFP RNA TRANSCRIPT:
AUGGUGAGCAAGGGCGAGGAGCUGUUCACCGGGGUGGUGCCCAUCCUG
GUCGAGCUGGACGGCGACGUAAACGGCCACAAGUUCAGCGUGUCCGGC
GAGGGCGAGGGCGAUGCCACCUACGGCAAGCUGACCCUGAAGUUCAUC
UGCACCACCGGCAAGCUGCCCGUGCCCUGGCCCACCCUCGUGACCACC
CUGACCUACGGCGUGCAGUGCUUCAGCCGCUACCCCGACCACAUGAAG
CAGCACGACUUCUUCAAGUCCGCCAUGCCCGAAGGCUACGUCCAGGAG
CGCACCAUCUUCUUCAAGGACGACGGCAACUACAAGACCCGCGCCGAG
GUGAAGUUCGAGGGCGACACCCUGGUGAACCGCAUCGAGCUGAAGGGC
AUCGACUUCAAGGAGGACGGCAACAUCCUGGGGCACAAGCUGGAGUAC
AACUACAACAGCCACAACGUCUAUAUCAUGGCCGACAAGCAGAAGAAC
GGCAUCAAGGUGAACUUCAAGAUCCGCCACAACAUCGAGGACGGCAGC
GUGCAGCUCGCCGACCACUACCAGCAGAACACCCCCAUCGGCGACGGC
CCCGUGCUGCUGCCCGACAACCACUACCUGAGCACCCAGUCCGCCCUG
AGCAAAGACCCCAACGAGAAGCGCGAUCACAUGGUCCUGCUGGAGUUC
GUGACCGCCGCCGGGAUCACUCUCGGCAUGGACGAGCUGUACAAGUAAA
ANTI-GFP
TARGET SITE: GCAAGCTGACCCTGAAGTTCAT

### A-siRNA:(RnU1->RISC).

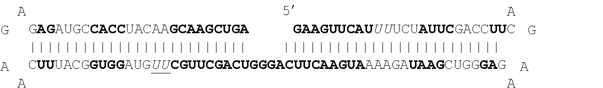

### A-shRNA: (RnU1->Dicer->RISC).

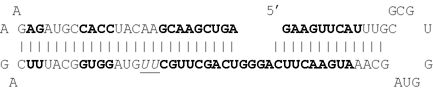

### Example 3

### Design and Production of Asymmetric Precursor Anti-Luciferase Polynucleotide

>U47296_m GL3
AUGGAAGACGCCAAAAACAUAAAGAAAGGCCCGGCGCCAUUCUAUCCG
CUGGAAGAUGGAACCGCUGGAGAGCAACUGCAUAAGGCUAUGAAGAGA
UACGCCCUGGUUCCUGGAACAAUUGCUUUUACAGAUGCACAUAUCGAG
GUGGACAUCACUUACGCUGAGUACUUCGAAAUGUCCGUUCGGUUGGCA
GAAGCUAUGAAACGAUAUGGGCUGAAUACAAAUCACAGAAUCGUCGUA
UGCAGUGAAAACUCUCUUCAAUUCUUUAUGCCGGUGUUGGGCGCGUUA
UUUAUCGGAGUUGCAGUUGCGCCCGCGAACGACAUUUAUAAUGAACGU
GAAUUGCUCAACAGUAUGGGCAUUUCGCAGCCUACCGUGGUGUUCGUU
UCCAAAAAGGGGUUGCAAAAAAUUUUGAACGUGCAAAAAAAGCUCCCA
AUCAUCCAAAAAAUUAUUAUCAUGGAUUCUAAAACGGAUUACCAGGGA
UUUCAGUCGAUGUACACGUUCGUCACAUCUCAUCUACCUCCCGGUUUU
AAUGAAUACGAUUUUGUGCCAGAGUCCUUCGAUAGGGACAAGACAAUU
GCACUGAUCAUGAACUCCUCUGGAUCUACUGGUCUGCCUAAAGGUGUC
GCUCUGCCUCAUAGAACUGCCUGCGUGAGAUUCUCGCAUGCCAGAGAU
CCUAUUUUUGGCAAUCAAAUCAUUCCGGAUACUGCGAUUUUAAGUGUU
GUUCCAUUCCAUCACGGUUUUGGAAUGUUUACUACACUCGGAUAUUUG
AUAUGUGGAUUUCGAGUCGUCUUAAUGUAUAGAUUUGAAGAAGAGCUG
UUUCUGAGGAGCCUUCAGGAUUACAAGAUUCAAAGUGCGCUGCUGGUG
CCAACCCUAUUCUCCUUCUUCGCCAAAAGCACUCUGAUUGACAAAUAC
GAUUUAUCUAAUUUACACGAAAUUGCUUCUGGUGGCGCUCCCCUCUCU
AAGGAAGUCGGGGAAGCGGUUGCCAAGAGGUUCCAUCUGCCAGGUAUC
AGGCAAGGAUAUGGGCUCACUGAGACUACAUCAGCUAUUCUGAUUACA
CCCGAGGGGGAUGAUAAACCGGGCGCGGUCGGUAAAGUUGUUCCAUUU
UUUGAAGCGAAGGUUGUGGAUCUGGAUACCGGGAAAACGCUGGGCGUU
AAUCAAAGAGGCGAACUGUGUGUGAGAGGUCCUAUGAUUAUGUCCGGU
UAUGUAAACAAUCCGGAAGCGACCAACGCCUUGAUUGACAAGGAUGGA
UGGCUACAUUCUGGAGACAUAGCUUACUGGGACGAAGACGAACACUUC
UUCAUCGUUGACCGCCUGAAGUCUCUGAUUAAGUACAAAGGCUAUCAG
GUGGCUCCCGCUGAAUUGGAAUCCAUCUUGCUCCAACACCCCAACAUC
UUCGACGCAGGUGUCGCAGGUCUUCCCGACGAUGACGCCGGUGAACUU
CCCGCCGCCGUUGUUGUUUUGGAGCACGGAAAGACGAUGACGGAAAAA
GAGAUCGUGGAUUACGUCGCCAGUCAAGUAACAACCGCGAAAAAGUUG
CGCGGAGGAGUUGUGUUUGUGGACGAAGUACCGAAAGGUCUUACCGGA
AAACUCGACGCAAGAAAAAUCAGAGAGAUCCUCAUAAAGGCCAAGAAG
GGCGGAAAGAUCGCCGUGUAA
Anti-Luc
Target Site: CUUACGCUGAGUACUUCGAAAU

### A-siRNA: Anti-Luc(Rnt1->RISC).

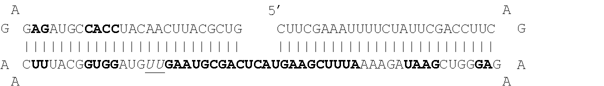

### A-shRNA: Anti-Luc(Rnt1->Dicer->RISC).

```
LOOP Gene BINDER =
      AAGAUAG GCGACCUU CUACCUU 5′
5′-UUCUAUC CGCUGGAA GAUGGAA . . . gene
      ((((((( ........ )))))))
```

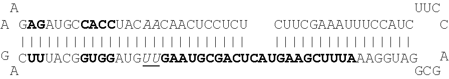

### Example 4

### Design and Production of Asymmetric Precursor Anti-Staph_MRSA Polynucleotide

### Staphylococcus Aureus Methicillin-Resistance/TST Suppression

The use of Asymmetric RNAi against is proposed to suppress MRSA resistance to methicillin by suppression of the Methicillin Resistance Gene (MecR1); gi|156720466:2139133-2139837. The Methicillin Resistance Regulatory Gene (Mecl) RNAi site may be substituted for MecR1. In one embodiment(#2), a secondary fragment is also designed to reduce the expression of Toxic Shock Protein (TST); gi|56720466:48855-49226.

METHYCILLIN-RESISTANCE GENE (MecR1):
ATGGATAATAAAACGTATGAAATATCATCTGCAGAATGGGAATTTATG
AATATCATTTGGATGAAAAAATATGCAAGTGCGAATAATATAATAGAA
GAAATACAAATGCAAAAGGACTGGAGTCCAAAAACCATTCGTACACTT
ATAACGAGATTGTATAAAAAGGGATTTATAGATCGTAAAAAAGACAAT
AAAATTTTTCAATATTACTCTCTTGTAGAAGAAAGTGATATAAAATAT
AAAACATCTAAAAACTTTATCAATAAAGTATACAAAGGCGGTTTCAAT
TCACTTGTCTTAAACTTTGTAGAAAAAGAAGATCTATCACAAGATGAA
ATAGAAGAATTGAGAAATATATTGAATAAAAAATAA
RNAi Site: TACAAAGGCGGTTTCAATTCAC
ASYMMETRIC MOTIF: GTGAATTGAAACCGCCTTTGTA,
LACKS DIMERIZATION
METHYCILLIN-RESISTANCE REGULATORY GENE (MecI):
GTGTTATCATCTTTTTTAATGTTAAGTATAATCAGTTCATTGCTCACG
ATATGTGTAATTTTTTTAGTGAGAATGCTCTATATAAAATATACTCAA
AATATTATGTCACATAAGATTTGGTTATTAGTGCTCGTCTCCACGTTA
ATTCCATTAATACCATTTTACAAAATATCGAATTTTACATTTTCAAAA
GATATGATGAATCGAAATGTATCTGACACGACTTCTTCGGTTAGTCAT
ATGTTAGATGGTCAACAATCATCTGTTACGAAAGACTTAGCAATTAAT
GTTAATCAGTTTGAGACCTCAAATATAACGTATATGATTCTTTTGATA
TGGGTATTTGGTAGTTTGTTGTGCTTATTTTATATGATTAAGGCATTC
CGACAAATTGATGTTATTAAAAGTTCGTCATTGGAATCGTCATATCTT
AATGAACGACTTAAAGTATGTCAAAGTAAGATGCAGTTCTACAAAAAG
CATATAACAATTAGTTATAGTTCAAACATTGATAATCCGATGGTATTT
GGTTTAGTGAAATCCCAAATTGTACTACCAACTGTCGTAGTCGAAACC
ATGAATGACAAAGAAATTGAATATATTATTCTACATGAACTATCACAT
GTGAAAAGTCATGACTTAATATTCAACCAGCTTTATGTTGTTTTTAAA
ATGATATTCTGGTTTAATCCTGCACTATATATAAGTAAAACAATGATG
GACAATGACTGTGAAAAAGTATGTGATAGAAACGTTTTAAAAATTTTG
AATCGCCATGAACATATACGTTATGGTGAATCGATATTAAAATGCTCT
ATTTTAAAATCTCAGCACATAAATAATGTGGCAGCACAATATTTACTA
GGTTTTAATTCAAATATTAAAGAACGTGTTAAGTATATTGCACTTTAT
GATTCAATGCCTAAACCTAATCGAAACAAGCGTATTGTTGCGTATATT
GTATGTAGTATATCGCTTTTAATACAAGCACCGTTACTATCTGCACAT
GTTCAACAAGACAAATATGAAACAAATGTATCATATAAAAAATTAAAT
CAACTAGCTCCGTATTTCAAAGGATTTGATGGAAGTTTTGTGCTTTAT
AATGAACGGGAGCAAGCTTATTCTATTTATAATGAACCAGAAAGTAAA
CAACGATATTCACCTAATTCTACTTACAAAATTTATTTAGCGTTAATG
GCATTCGACCAAAATTTACTCTCATTAAATCATACTGAACAACAATGG
GATAAACATCAATATCCATTTAAAGAATGGAACCAAGATCAAAATTTA
AATTCTTCAATGAAATATTCAGTAAATTGGTATTACGAAAATTTAAAC
AAACATTTAAGACAAGATGAGGTTAAATCTTATTTAGATCTAATTGAA
TATGGTAATGAAGAAATATCAGGGAATGAAAATTATTGGAATGAATCT
TCATTAAAAATTTCTGCAATAGAACAGGTTAATTTGTTGAAAAATATG
AAACAACATAACATGCATTTTGATAATAAGGCTATTGAAAAAGTTGAA
AATAGTATGACTTTGAAACAAAAAGATACTTATAAATATGTAGGTAAA
ACTGGAACAGGAATCGTGAATCACAAAGAAGCAAATGGATGGTTCGTA
GGTTATGTTGAAACGAAAGATAATACGTATTATTTTGCTACACATTTA
AAAGGCGAAGACAATGCGAATGGCGAAAAAGCACAACAAATTTCTGAG
CGTATTTTAAAAGAAATGGAGTTAATATAA
RNAi Site: TAAATCAACTAGCTCCGTA, 0 homology to
human AYSMMETRIC RNAi Motif:
UACGGAGCUAGUUGAUUUA . . . (0.00)
TOXIC SHOCK GENE (TST):
ATGAATAAAAAATTACTAATGAATTTTTTTATCGTAAGCCCTTTGTTG
CTTGCGACAATCGCTACAGATTTTACCCCTGTTCCCTTATCATCTAAT
CAAATAATCAAAACTGCAAAAGCATCTACAAACGATAATATAAAGGAT
TTGCTAGACTGGTATAGTAGTGGGTCTGACACTTTTACAAATAGTGAA
GTTTTAGATAATTCCTTAGGATCTATGCGTATAAAAAACACAGATGGC
AGCATCAGCCTTATAATTTTTCCGAGTCCTTATTATAGCCCTGCTTTT
ACAAAAGGGGAAAAAGTTGACTTAAACACAAAAAGAACTAAAAAAAGC
CAACATACTAGCGAAGGAACTTATATCCATTTCCAAATAAGTGGCGTT
ACAAATACTGAAAAATTACCTACTCCAATAGAACTACCTTTAAAAGTT
AAGGTTCATGGTAAAGATAGCCCCTTAAAGTATTGGCCAAAGTTCGAT
AAAAAACAATTAGCTATATCAACTTTAGACTTTGAAATTCGTCATCAG
CTAACTCAAATACATGGATTATATCGTTCAAGCGATAAAACGGGTGGT
TATTGGAAAATAACAATGAATGACGGATCCACATATCAAAGTGATTTA
TCTAAAAAGTTTGAATACAATACTGAAAAACCACCTATAAATATTGAT
GAAATAAAAACTATAGAAGCAGAAATTAATTAA
MRSA Target Site: TACAAAGGCGGTTTCAATTCAC
TST-secondary target Site:
TCGTAAGCCCTTTGTTGCTTGCGA

### Targeted as Antisense Naturally Occurring Stem-Loop:

AGCAUUCGGGAAACAACGAACGCU-5′
5′-ATGAATAAAAAATTACTAATGAATTTTTTTATCGTAAGCCCTTTGTTGCTTGCGACAATCGCT = gene
5′-................((((.((((((....((((((((........)))))))).....((( = fold notation

### Targeting Molecules:

### A-siRNA:(Rnt1->RISC).

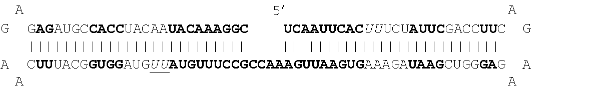

### A-shRNA: Anti-MRSA/TST (Rnt1->Dicer->RISC).

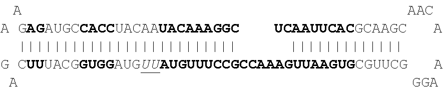

### Example 5

### Design and Production of Asymmetric Precursor Anti-HIV NEF Polynucleotide

HIV_NEF TARGET SITE: UGUGCCUGGCUAGAAGCACAAG

### A-siRNA:(Rnt1->RISC).

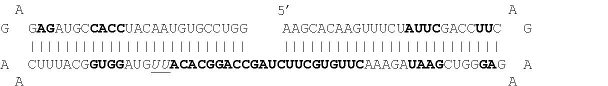

### A-shRNA: (Rnt1->Dicer->RISC).

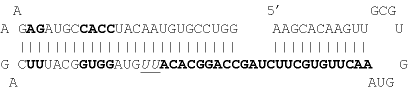

### Example 6

### Design and Production of Asymmetric Precursor Anti-P53 Polynucleotide

>AB082923
CGUGCUUUCCACGACGGUGACACGCUUCCCUGGAUUGGCCAGACUGC
CUUCCGGGUCACUGCCAUGGAGGAGCCGCAGUCAGAUCCUAGCGUCG
AGCCCCCUCUGAGUCAGGAAACAUUUUCAGACCUAUGGAAACUACUUC
CUGAAAACAACGUUCUGUCCCCCUUGCCGUCCCAAGCAAUGGAUGAUU
UGAUGCUGUCCCCGGACGAUAUUGAACAAUGGUUCACUGAAGACCCAG
GUCCAGAUGAAGCUCCCAGAAUGCCAGAGGCUGCUCCCCGCGUGGCC
CCUGCACCAGCAGCUCCUACACCGGCGGCCCCUGCACCAGCCCCCUC
CUGGCCCCUGUCAUCUUCUGUCCCUUCCCAGAAAACCUACCAGGGCA
GCUACGGUUUCCGUCUGGGCUUCUUGCAUUCUGGGACAGCCAAGUCU
GUGACUUGCACGUACUCCCCUGCCCUCAACAAGAUGUUUUGCCAACU
GGCCAAGACCUGCCCUGUGCAGCUGUGGGUUGAUUCCACACCCCCGC
CCGGCACCCGCGUCCGCGCCAUGGCCAUCUACAAGCAGUCACAGCAC
AUGACGGAGGUUGUGAGGCGCUGCCCCCACCAUGAGCGCUGCUCAGA
UAGCGAUGGUCUGGCCCCUCCUCAGCAUCUUAUCCGAGUGGAAGGAA
AUUUGCGUGUGGAGUAUUUGGAUGACAGAAACACUUUUCGACAUAGU
GUGGUGGUGCCCUAUGAGCCGCCUGAGGUUGGCUCUGACUGUACCAC
CAUCCACUACAACUACAUGUGUAACAGUUCCUGCAUGGGCGGCAUGAA
CCGGAGGCCCAUCCUCACCAUCAUCACACUGGAAGACUCCAGUGGUAA
UCUACUGGGACGGAACAGCUUUGAGGUGCAUGUUUGUGCCUGUCCUG
GGAGAGACCGGCGCACAGAGGAAGAGAAUCUCCGCAAGAAAGGGGAG
CCUCACCACGAGCUGCCCCCAGGGAGCACUAAGCGAGCACUGUCCAAC
AACACCAGCUCCUCUCCCCAGCCAAAGAAGAAACCACUGGAUGGAGAA
UAUUUCACCCUUCAGAUCCGUGGGCGUGAGCGCUUCGAGAUGUUCCG
AGAGCUGAAUGAGGCCUUGGAACUCAAGGAUGCCCAGGCUGGGAAGG
AGCCAGGGGGGAGCAGGGCUCACUCCAGCCACCUGAAGUCCAAAAAG
GGUCAGUCUACCUCCCGCCAUAAAAAACUCAUGUUCAAGACAGAAGGG
CCUGACUCAGACUGACAUUCUCCACUUCUUGUUCCCCACUGACAGCCU
CCCACCCCCAUCUCUCCCUCCCCUGCCAUUUUGGGUUUUGGGUCUUU
GAACCCUUGCUUGCAAUAGGUGUGCGUCAGAAGCACCCAGGACUUCC
AUUUGCUUUGUCCCGGGGCUCCACUGAACAAGUUGGCCUGCACUGGU
GUUUUGUUGUGGGGAGGAGGAUGGGGAGUAGGACAUACCAGCUUAGA
UUUUAAGGUUUUUACUGUGAGGGAUGUUUGGGAGAUGUAAGAAAUGU
UCUUGCAGUUAAGGGUUAGUUUACAAUCAGCCACAUUCUAGGUAGGG
GCCCACUUCACCGUACUAACCAGGGAAGCUGUCCCUCACUGUUGAAUU
UUCUCUAACUUCAAGGCCCAUAUCUGUGAAAUGCUGGCAUUUGCACCU
ACCUCACAGAGUGCAUUGUGAGGGUUAAUGAAAUAAUGUACAUCUGGC
CUUGAAACCACCUUUUAUUACAUGGGGUCUAGAACUUGACCCCCUUGA
GGGUGCUUGUUCCCUCUCCCUGUUGGUCGGUGGGUUGGUAGUUUCU
ACAGUUGGGCAGCUGGUUAGGUAGAGGGAGUUGUCAAGUCUCUGCUG
GCCCAGCCAAACCCUGUCUGACAACCUCUUGGUGAACCUUAGUACCUA
AAAGGAAAUCUCACCCCAUCCCACACCCUGGAGGAUUUCAUCUCUUGU
AUAUGAUGAUCUGGAUCCACCAAGACUUGUUUUAUGCUCAGGGUCAAU
UUCUUUUUUCUUUUUUUUUUUUUUUUUCUUUUUCUUUGAGACUGGGU
CUCGCUUUGUUGCCCAGGCUGGAGUGGAGUGGCGUGAUCUUGGCUUA
CUGCAGCCUUUGCCUCCCCGGCUCGAGCAGUCCUGCCUCAGCCUCCG
GAGUAGCUGGGACCACAGGUUCAUGCCACCAUGGCCAGCCAACUUUU
GCAUGUUUUGUAGAGAUGGGGUCUCACAGUGUUGCCCAGGCUGGUCU
CAAACUCCUGGGCUCAGGCGAUCCACCUGUCUCAGCCUCCCAGAGUG
CUGGGAUUACAAUUGUGAGCCACCACGUCCAGCUGGAAGGGUCAACA
UCUUUUACAUUCUGCAAGCACAUCUGCAUUUUCACCCCACCCUUCCCC
UCCUUCUCCCUUUUUAUAUCCCAUUUUUAUAUCGAUCUCUUAUUUUAC
AAUAAAACUUUGCUGCCAAAAAAAAAAAAAAAAAAAA
TARGET SITE: CCCUGCCCUCAACAAGAUGUUU

### A-siRNA:(Rnt1->RISC).

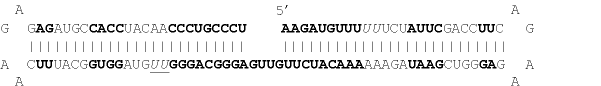

### A-shRNA: (Rnt1->Dicer->RISC).

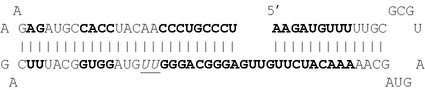

### REFERENCES

1. Fire, A., Xu, S., Montgomery, M. K., Kostas, S. A., Driver, S. E., and Mello, C. C. (1998) Potent and specific genetic interference by double stranded RNA in Caenorhabditis elegans. Nature. 408, 325-330.
2. Kennerdell, J. R., and Carthew, R. W. (1998) Use of dsRNA-mediated genetic interference to demonstrate that frizzled and frizzled 2 act in the wingless pathway. Cell. 95, 1017-1026.
3. Misquitta, L., and Paterson, B. M. (1999) Targeted disruption of gene function in Drosophila by RNA interference (RNA-i): a role for nautilus in embryonic somatic muscle formation. Proc. Natl. Acad. Sci. USA. 96, 1451-1456.
4. Hammond, S. M., Bernstein, E., Beach, D., and Hannon, G. J. (2000) An RNA-directed nuclease mediates post transcriptional gene silencing in Drosophila cells. Nature. 404, 293-296.
5. Lohmann, J. U., Endl, I., and Bosch, T. C. (1999) Silencing of developmental genes in Hydra. Dev. Biol. 214, 211-214.
6. Wargelius, A., Ellingsen, S., and Fjose, A. (1999) Double stranded RNA induces specific developmental defects in zebrafish embyos. Biochem. Biophys. Res. Commun. 263, 156-161.
7. Ngo, H., Tschudi, C., Gull, K., and Ullu, E. (1998) Double stranded RNA induces gene degradation in Trypanosoma brucei. Proc. Natl. Acad. Sci. USA. 95, 14687-14692.
8. Montgomery, M. K., Xu, S., Fire, A. (1998) RNA as a target of double stranded RNA mediated genetic interference in Caenorhabiditis elegans. Proc. Natl. Acad. Sci. USA. 95, 15502-15507.
9. Bosher, J. M., Dufourcq, P., Sookhareea, S., Labouesse, M. (1999) RNA interference can target pre-gene. Consequences for gene expression in Caenorhabiditis elegans operon. Genetics. 153, 1245-1256.
10. Fire, A. (1999) RNA-triggered gene silencing. Trends Genet. 15, 358-363.
11. Sharp, P. A. (1999) RNAi and double-stranded RNA. Genes Dev. 13, 139-141.
12. Ketting, R. F., Harerkamp, T. H., van Luenen, H. G., and Plasterk, R. H. (1999) Mut-7 of C. elegans, required for transposon silencing and RNA interference, is a homolog of Werner syndrome helicase and RNase I. Cell. 99, 133-141.
13. Tabara, H., Sarkissian, M., Kelly, W. G., Fleenor, J., Grishok, A., Timmons, L., Fire, A., and Mello, C. C. (1999) The rde-1 gene, RNA interference, and transposon silencing in C. elegans. Cell. 99, 123-132.
14. Zamore, P. D., Tuschl, T., Sharp, P. A., and Bartel, D. P. (2000) RNAi: Double stranded RNA directs the ATP dependent cleavage of gene at 21 to 23 nucleotide intervals. Cell. 101, 25-33.
15. Bernstein, E., Caudy, A. A., Hammond, S. M., and Hannon, G. J. (2001) Role for a bidentate ribonuclease in the initiation step of RNA interference. Nature. 409, 363-366.
16. Elbashir, S., Lendeckel, W., and Tuschl, T. (2001) RNA interference is mediated by 21 and 22 nucleotide RNAs. Genes and Dev. 15, 188-200.
17. Sharp, P. A. (2001) RNA interference 2001. Genes and Dev. 15, 485-490.
18. Hunter, T., Hunt, T., and Jackson, R. J. (1975) The characteristics of inhibition of protein synthesis by double-stranded ribonucleic acid in reticulocyte lysates. J. Biol. Chem. 250, 409-417.
19. Bass, B. L. (2001) The short answer. Nature. 411, 428-429.
20. Elbashir, S. M., Harborth, J., Lendeckel, W., Yalcin, A., Weber, K., and Tuschl, T. (2001) Duplexes of 21-nucleotide RNAs mediate RNA interference in cultured mammalian cells. Nature. 411, 494-498.
21. Carson, P. E. and Frischer, H. (1966) Glucose-6-Phosphate dehydrogenase deficiency and related disorders of the pentose phosphate pathway. Am J. Med. 41, 744-764.
22. Stamato, T. D., Mackenzie, L., Pagani, J. M., and Weinstein, R. (1982) Mutagen treatment of single Chinese Hamster Ovary cells produce colonies mosaic for Glucose-6-phosphate dehydrogenase activity. Somatic Cell Genetics. 8, 643-651.
23. Genetic characterization of methicillin-resistant Staphylococcus aureus Vaccine. 2004, Dec. 6; 22 Suppl 1:S5-8. [Hiramatsu K, Watanabe S, Takeuchi F, Ito T, and Baba T]

1. An isolated, self-forming precursor polynucleotide, comprising:
(a) a targeting region comprising a polynucleotide sequence complementary to a region of a target gene sequence;
(b) a first self-complementary region; and
(c) a second self-complementary region,
wherein the first and second self-complementary regions are located one at each end of the targeting region and both self-complementary regions form stem-loop structures, wherein the first self-complementary region is capable of being cleaved by a RNase III endoribonuclease that is not a class IV DICER endoribonuclease, and
wherein both self-complementary regions comprise a nucleotide sequence that is complementary to a region of the target gene sequence, but wherein a portion of the target sequence present in the targeting region does not have a complementary sequence in either of the self-complementary regions.

2. The polynucleotide of claim 1, wherein the polynucleotide sequence complementary to the region of the target gene sequence of (a) consists of about 17 to about 30 nucleotides in length.

3. The polynucleotide of claim 1, wherein both the first and second self-complementary regions form stem-loop structures capable of being cleaved by a RNase III endoribonuclease that is not a class IV DICER endoribonuclease.

4. The polynucleotide of claim 1, comprising one or more of the following:
(a) the self-complementary regions that form stem-loop structures capable of being cleaved by a RNase III endoribonuclease that is not a class IV DICER endoribonuclease are about 28 to about 54 nucleotides in length;
(b) the stem-loop structure of the self-complementary regions capable of being cleaved by a RNase III endoribonuclease that is not a class IV DICER endoribonuclease comprise loops consisting of 2-6 nucleotides;
(c) the stem-loop structure of the self-complementary regions capable of being cleaved by a RNase III endoribonuclease that is not a class IV DICER endoribonuclease comprise tetraloops consisting of a nucleotide sequence selected from NGNN and AAGU;
(d) the stem-loop structure of the second self-complementary region comprises a loop structure of 5 nucleotides to about 14 nucleotides and is resistant to cleavage by a RNase III endoribonuclease that is not a class IV DICER endoribonuclease
(e) the second self-complementary region is capable of being cleaved by a class IV DICER endoribonuclease; and
(f) the second self-complementary region is about 9 to about 20 nucleotides in length.

5.-10. (canceled)

11. The polynucleotide of claim 1, wherein the polynucleotide is capable of being processed in a cell to form a biologically active asymmetric siRNA or miRNA polynucleotide.

12.-14. (canceled)

15. The polynucleotide of claim 1, wherein said polynucleotide is RNA.

16. An isolated polynucleotide comprising from 5′ to 3′:
(a) a first self-complementary region capable of forming a stem-loop structure;
(b) a targeting region comprising a polynucleotide sequence reverse complementary to a region of a target gene sequence; and
(c) a second self-complementary region capable of forming a stem-loop structure,
wherein at least one of the first or second self-complementary regions is capable of being cleaved by a RNase III endoribonuclease that is not a class IV DICER endoribonuclease, and
wherein both the first and second self-complementary regions comprise a nucleotide sequence that is complementary to a region of the target gene sequence, but wherein a portion of the target sequence present in the targeting region does not have a complementary sequence in either of the self-complementary regions.

17. The polynucleotide of claim 16, wherein the polynucleotide sequence reverse complementary to the region of the target gene sequence of (b) consists of about 17 to about 30 nucleotides in length.

18. The polynucleotide of claim 16, comprising one or more of the following:
(a) both self-complementary regions form stem-loop structures capable of being cleaved by a RNase III endoribonuclease that is not a class IV DICER endoribonuclease;
(b) the self-complementary regions that form stem-loop structures capable of being cleaved by a RNase III endoribonuclease that is not a class IV DICER endonuclease is about 28 to about 54 nucleotides in length;
(c) the stem-loop structure of the self-complementary regions capable of being cleaved by a RNase III endoribonuclease that is not a class IV DICER endoribonuclease comprise loops consisting of 2-6 nucleotides; and
(d) the stem-loop structure of the self-complementary regions capable of being cleaved by a RNase III endoribonuclease that is not a class IV DICER endoribonuclease comprise tetraloops consisting of a nucleotide sequence selected from NGNN and AAGU.

19.-21. (canceled)

22. The polynucleotide of claim 16, wherein one of the first or second self-complementary regions forms a stem-loop structure capable of being cleaved by a RNase III endoribonuclease that is not a class IV DICER endoribonuclease, and the other self-complementary region comprises a loop structure of 5 nucleotides to about 14 nucleotides and is resistant to cleavage by a RNase III endoribonuclease that is not a class IV DICER endoribonuclease.

23. The polynucleotide of claim 22, wherein the self-complementary region comprising a loop structure of 5 nucleotides to about 14 nucleotides:
(a) comprises nucleotides capable of forming about 5-14 complementary base pairs having a helical structure;
(b) is capable of being cleaved by a class IV DICER endoribonuclease; or
(c) is about 9 to about 20 nucleotides in length.

24.-25. (canceled)

26. The polynucleotide of claim 16, wherein the polynucleotide is capable of being processed in a cell to form a biologically active asymmetric siRNA or miRNA polynucleotide.

27. The polynucleotide of claim 16, wherein said polynucleotide is RNA or double-stranded DNA.

28. (canceled)

29. An expression vector capable of producing a polynucleotide of claim 1 or claim 16.

30. (canceled)

31. A host cell comprising the expression vector of claim 29 or 30.

32. A method of inhibiting or reducing expression of a target gene in a cell, comprising introducing a polynucleotide of claim 1 or claim 16 into the cell, thereby inhibiting or reducing expression of the target gene in the cell.

33. The method of claim 32, wherein:
(a) the polynucleotide is processed in the cell by a RNase III endoribonuclease that is not a class IV DICER endoribonuclease to form a biologically active asymmetric siRNA or miRNA polynucleotide; or
(b) the polynucleotide is processed in the cell by a RNase III endoribonuclease that is not a class IV DICER endoribonuclease and a class IV DICER endoribonuclease to form a biologically active asymmetric siRNA or miRNA polynucleotide.

34. (canceled)

35. A method of inhibiting or reducing expression of a target gene in a cell, comprising introducing an expression vector of claim 29 into the cell, thereby inhibiting or reducing expression of the target gene in the cell.

36. The method of claim 35, wherein:
(a) the polynucleotide produced by the expression vector is processed in the cell by a RNase III endoribonuclease that is not a class IV DICER endoribonuclease to form a biologically active asymmetric siRNA or miRNA polynucleotide; or
(b) the polynucleotide produced by the expression vector is processed in the cell by a RNase III endoribonuclease that is not a class IV DICER endoribonuclease and a class IV DICER endonuclease to form a biologically active asymmetric siRNA or miRNA polynucleotide.

37. (canceled)


SEQUENCE LISTING (38 sequences)

SEQ ID NO: 1 - DNA, 22 bp, Artificial Sequence
  Design part for assymetric siRNA molecule
nnnnnnnnnnnnnnnnnnnnnn

SEQ ID NO: 2 - RNA, 34 nt, Artificial Sequence
  Design part for assymetric siRNA molecule - 5′ stem loop
guagguggcauuucagaagagaugccaccuacaa

SEQ ID NO: 3 - RNA, 34 nt, Artificial Sequence
  Design part for assymetric siRNA molecule - 3′ stem loop
ucuauucgaccuucagaagagggucgaauagaaa

SEQ ID NO: 4 - RNA, 16 nt, Artificial Sequence
  Design part for assymetric shRNA molecule - shRNA stem loop
uugcgcgugguagcaa

SEQ ID NO: 5 - RNA, 112 nt, Artificial Sequence
  Assymetric siRNA molecule
nnnnnnnnnuuucuauucgaccuucagaagagggucgaauagaaannnnnnnnnnnnnnnnnnnnnnuuguagguggcauuucagaagagaugccaccuacaannnnnnnnn

SEQ ID NO: 6 - RNA, 92 nt, Artificial Sequence
  Assymetric shRNA molecule
nnnnnnnnnuugcgcgugguagcaannnnnnnnnnnnnnnnnnnnnnuuguagguggcauuucagaagagaugccaccuacaannnnnnnnn

SEQ ID NO: 7 - DNA, 62 bp, Artificial Sequence
  Positive control shRNA sequence
gatccccgcaagctgaccctgaagttcttcaagagagaacttcagggtcagcttgcttttta

SEQ ID NO: 8 - DNA, 62 bp, Artificial Sequence
  Positive control shRNA sequence
agcttaaaaagcaagctgaccctgaagttctctcttgaagaacttcagggtcagcttgcggg

SEQ ID NO: 9 - DNA, 60 bp, Artificial Sequence
  Negative control shRNA sequence
gatccccgcgcgctttgtaggattcgttcaagagacgaatcctacaaagcgcgcttttta

SEQ ID NO: 10 - DNA, 60 bp, Artificial Sequence
  Negative control shRNA sequence
agcttaaaaagcgcgctttgtaggattcgtctcttgaacgaatcctacaaagcgcgcggg

SEQ ID NO: 11 - DNA, 721 bp, Artificial Sequence
  EGFP sequence obtained from pIRES-EGFP vector
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaa

SEQ ID NO: 12 - RNA, 721 nt, Artificial Sequence
  RNA transcript of EGFP sequence obtained from pIRES-EGFP vector
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguaaa

SEQ ID NO: 13 - DNA, 22 bp, Artificial Sequence
  Anti-GFP target sequence
gcaagctgaccctgaagttcat

SEQ ID NO: 14 - RNA, 112 nt, Artificial Sequence
  GFP assymetric siRNA molecule
gaaguucauuuucuauucgaccuucagaagagggucgaauagaaaaugaacuucagggucagcuugcuuguagguggcauuucagaagagaugccaccuacaagcaagcuga

SEQ ID NO: 15 - RNA, 92 nt, Artificial Sequence
  GFP assymetric shRNA molecule
gaaguucauuugcgcgugguagcaaaugaacuucagggucagcuugcuuguagguggcauuucagaagagaugccaccuacaagcaagcuga

SEQ ID NO: 16 - RNA, 1653 nt, Artificial Sequence
  Luciferase sequence from cloning vector pGL3
auggaagacgccaaaaacauaaagaaaggcccggcgccauucuauccgcuggaagauggaaccgcuggagagcaacugcauaaggcuaugaagagauacgcccugguuccuggaacaauugcuuuuacagaugcacauaucgagguggacaucacuuacgcugaguacuucgaaauguccguucgguuggcagaagcuaugaaacgauaugggcugaauacaaaucacagaaucgucguaugcagugaaaacucucuucaauucuuuaugccgguguugggcgcguuauuuaucggaguugcaguugcgcccgcgaacgacauuuauaaugaacgugaauugcucaacaguaugggcauuucgcagccuaccgugguguucguuuccaaaaagggguugcaaaaaauuuugaacgugcaaaaaaagcucccaaucauccaaaaaauuauuaucauggauucuaaaacggauuaccagggauuucagucgauguacacguucgucacaucucaucuaccucccgguuuuaaugaauacgauuuugugccagaguccuucgauagggacaagacaauugcacugaucaugaacuccucuggaucuacuggucugccuaaaggugucgcucugccucauagaacugccugcgugagauucucgcaugccagagauccuauuuuuggcaaucaaaucauuccggauacugcgauuuuaaguguuguuccauuccaucacgguuuuggaauguuuacuacacucggauauuugauauguggauuucgagucgucuuaauguauagauuugaagaagagcuguuucugaggagccuucaggauuacaagauucaaagugcgcugcuggugccaacccuauucuccuucuucgccaaaagcacucugauugacaaauacgauuuaucuaauuuacacgaaauugcuucugguggcgcuccccucucuaaggaagucggggaagcgguugccaagagguuccaucugccagguaucaggcaaggauaugggcucacugagacuacaucagcuauucugauuacacccgagggggaugauaaaccgggcgcggucgguaaaguuguuccauuuuuugaagcgaagguuguggaucuggauaccgggaaaacgcugggcguuaaucaaagaggcgaacugugugugagagguccuaugauuauguccgguuauguaaacaauccggaagcgaccaacgccuugauugacaaggauggauggcuacauucuggagacauagcuuacugggacgaagacgaacacuucuucaucguugaccgccugaagucucugauuaaguacaaaggcuaucagguggcucccgcugaauuggaauccaucuugcuccaacaccccaacaucuucgacgcaggugucgcaggucuucccgacgaugacgccggugaacuucccgccgccguuguuguuuuggagcacggaaagacgaugacggaaaaagagaucguggauuacgucgccagucaaguaacaaccgcgaaaaaguugcgcggaggaguuguguuuguggacgaaguaccgaaaggucuuaccggaaaacucgacgcaagaaaaaucagagagauccucauaaaggccaagaagggcggaaagaucgccguguaa

SEQ ID NO: 17 - RNA, 22 nt, Artificial Sequence
  Anti-luciferase target sequence
cuuacgcugaguacuucgaaau

SEQ ID NO: 18 - RNA, 112 nt, Artificial Sequence
  Anti-luciferase asymmetric siRNA molecule
cuucgaaauuuucuauucgaccuucagaagagggucgaauagaaaauuucgaaguacucagcguaaguuguagguggcauuucagaagagaugccaccuacaacuuacgcug

SEQ ID NO: 19 - RNA, 99 nt, Artificial Sequence
  Anti-luciferase asymmetric shRNA molecule
cuucgaaauuuccaucuuccagcggauggaaauuucgaaguacucagcguaaguuguagguggcauuucagaagagaugccaccuacaacaacuccucu

SEQ ID NO: 20 - DNA, 372 bp, Staphylococcus aureus
atggataataaaacgtatgaaatatcatctgcagaatgggaatttatgaatatcatttggatgaaaaaatatgcaagtgcgaataatataatagaagaaatacaaatgcaaaaggactggagtccaaaaaccattcgtacacttataacgagattgtataaaaagggatttatagatcgtaaaaaagacaataaaatttttcaatattactctcttgtagaagaaagtgatataaaatataaaacatctaaaaactttatcaataaagtatacaaaggcggtttcaattcacttgtcttaaactttgtagaaaaagaagatctatcacaagatgaaatagaagaattgagaaatatattgaataaaaaataa

SEQ ID NO: 21 - DNA, 22 bp, Staphylococcus aureus
  Asymmetric motif
tacaaaggcggtttcaattcac

SEQ ID NO: 22 - DNA, 22 bp, Artificial Sequence
  Asymmetric motif
gtgaattgaaaccgcctttgta

SEQ ID NO: 23 - DNA, 1758 bp, Staphylococcus aureus
gtgttatcatcttttttaatgttaagtataatcagttcattgctcacgatatgtgtaatttttttagtgagaatgctctatataaaatatactcaaaatattatgtcacataagatttggttattagtgctcgtctccacgttaattccattaataccattttacaaaatatcgaattttacattttcaaaagatatgatgaatcgaaatgtatctgacacgacttcttcggttagtcatatgttagatggtcaacaatcatctgttacgaaagacttagcaattaatgttaatcagtttgagacctcaaatataacgtatatgattcttttgatatgggtatttggtagtttgttgtgcttattttatatgattaaggcattccgacaaattgatgttattaaaagttcgtcattggaatcgtcatatcttaatgaacgacttaaagtatgtcaaagtaagatgcagttctacaaaaagcatataacaattagttatagttcaaacattgataatccgatggtatttggtttagtgaaatcccaaattgtactaccaactgtcgtagtcgaaaccatgaatgacaaagaaattgaatatattattctacatgaactatcacatgtgaaaagtcatgacttaatattcaaccagctttatgttgtttttaaaatgatattctggtttaatcctgcactatatataagtaaaacaatgatggacaatgactgtgaaaaagtatgtgatagaaacgttttaaaaattttgaatcgccatgaacatatacgttatggtgaatcgatattaaaatgctctattttaaaatctcagcacataaataatgtggcagcacaatatttactaggttttaattcaaatattaaagaacgtgttaagtatattgcactttatgattcaatgcctaaacctaatcgaaacaagcgtattgttgcgtatattgtatgtagtatatcgcttttaatacaagcaccgttactatctgcacatgttcaacaagacaaatatgaaacaaatgtatcatataaaaaattaaatcaactagctccgtatttcaaaggatttgatggaagttttgtgctttataatgaacgggagcaagcttattctatttataatgaaccagaaagtaaacaacgatattcacctaattctacttacaaaatttatttagcgttaatggcattcgaccaaaatttactctcattaaatcatactgaacaacaatgggataaacatcaatatccatttaaagaatggaaccaagatcaaaatttaaattcttcaatgaaatattcagtaaattggtattacgaaaatttaaacaaacatttaagacaagatgaggttaaatcttatttagatctaattgaatatggtaatgaagaaatatcagggaatgaaaattattggaatgaatcttcattaaaaatttctgcaatagaacaggttaatttgttgaaaaatatgaaacaacataacatgcattttgataataaggctattgaaaaagttgaaaatagtatgactttgaaacaaaaagatacttataaatatgtaggtaaaactggaacaggaatcgtgaatcacaaagaagcaaatggatggttcgtaggttatgttgaaacgaaagataatacgtattattttgctacacatttaaaaggcgaagacaatgcgaatggcgaaaaagcacaacaaatttctgagcgtattttaaaagaaatggagttaatataa

SEQ ID NO: 24 - RNA, 19 nt, Artificial Sequence
  Asymmetric RNAi motif
uacggagcuaguugauuua

SEQ ID NO: 25 - DNA, 705 bp, Unknown
  Unknown species for Toxic Shock Gene
atgaataaaaaattactaatgaatttttttatcgtaagccctttgttgcttgcgacaatcgctacagattttacccctgttcccttatcatctaatcaaataatcaaaactgcaaaagcatctacaaacgataatataaaggatttgctagactggtatagtagtgggtctgacacttttacaaatagtgaagttttagataattccttaggatctatgcgtataaaaaacacagatggcagcatcagccttataatttttccgagtccttattatagccctgcttttacaaaaggggaaaaagttgacttaaacacaaaaagaactaaaaaaagccaacatactagcgaaggaacttatatccatttccaaataagtggcgttacaaatactgaaaaattacctactccaatagaactacctttaaaagttaaggttcatggtaaagatagccccttaaagtattggccaaagttcgataaaaaacaattagctatatcaactttagactttgaaattcgtcatcagctaactcaaatacatggattatatcgttcaagcgataaaacgggtggttattggaaaataacaatgaatgacggatccacatatcaaagtgatttatctaaaaagtttgaatacaatactgaaaaaccacctataaatattgatgaaataaaaactatagaagcagaaattaattaa

SEQ ID NO: 26 - DNA, 22 bp, Staphylococcus aureus
tacaaaggcggtttcaattcac

SEQ ID NO: 27 - DNA, 24 bp, Unknown
  Unknown species for Toxic Shock Gene target sequence
tcgtaagccctttgttgcttgcga

SEQ ID NO: 28 - RNA, 24 nt, Unknown
  Unknown species for Toxic Shock Gene naturally occurring stem loop
agcauucgggaaacaacgaacgcu

SEQ ID NO: 29 - DNA, 63 bp, Unknown
  Unknown species for Toxic Shock Gene
atgaataaaaaattactaatgaatttttttatcgtaagccctttgttgcttgcgacaatcgct

SEQ ID NO: 30 - RNA, 112 nt, Artificial Sequence
  Anit-MRSA/TST asymmetric siRNA molecule
ucaauucacuuucuauucgaccuucagaagagggucgaauagaaagugaauugaaaccgccuuuguauuguagguggcauuucagaagagaugccaccuacaauacaaaggc

SEQ ID NO: 31 - RNA, 96 nt, Artificial Sequence
  Anit-MRSA/TST asymmetric shRNA molecule
ucaauucacgcaagcaacaaagggcuugcgugaauugaaaccgccuuuguauuguagguggcauuucagaagagaugccaccuacaauacaaaggc

SEQ ID NO: 32 - RNA, 22 nt, Human Immunodeficiency Virus
ugugccuggcuagaagcacaag

SEQ ID NO: 33 - RNA, 112 nt, Artificial Sequence
  Anit-HIV nef asymmetric siRNA molecule
aagcacaaguuucuauucgaccuucagaagagggucgaauagaaacuugugcuucuagccaggcacauuguagguggcauuucagaagagaugccaccuacaaugugccugg

SEQ ID NO: 34 - RNA, 88 nt, Artificial Sequence
  Anit-HIV nef asymmetric shRNA molecule
aagcacaaguugcgugguaaacuugugcuucuagccaggcacauuguagguggcauuucagaagagaugccaccuacaaugugccugg

SEQ ID NO: 35 - RNA, 2451 nt, Homo sapiens
cgugcuuuccacgacggugacacgcuucccuggauuggccagacugccuuccgggucacugccauggaggagccgcagucagauccuagcgucgagcccccucugagucaggaaacauuuucagaccuauggaaacuacuuccugaaaacaacguucugucccccuugccgucccaagcaauggaugauuugaugcuguccccggacgauauugaacaaugguucacugaagacccagguccagaugaagcucccagaaugccagaggcugcuccccgcguggccccugcaccagcagcuccuacaccggcggccccugcaccagcccccuccuggccccugucaucuucugucccuucccagaaaaccuaccagggcagcuacgguuuccgucugggcuucuugcauucugggacagccaagucugugacuugcacguacuccccugcccucaacaagauguuuugccaacuggccaagaccugcccugugcagcuguggguugauuccacacccccgcccggcacccgcguccgcgccauggccaucuacaagcagucacagcacaugacggagguugugaggcgcugcccccaccaugagcgcugcucagauagcgauggucuggccccuccucagcaucuuauccgaguggaaggaaauuugcguguggaguauuuggaugacagaaacacuuuucgacauagugugguggugcccuaugagccgccugagguuggcucugacuguaccaccauccacuacaacuacauguguaacaguuccugcaugggcggcaugaaccggaggcccauccucaccaucaucacacuggaagacuccagugguaaucuacugggacggaacagcuuugaggugcauguuugugccuguccugggagagaccggcgcacagaggaagagaaucuccgcaagaaaggggagccucaccacgagcugcccccagggagcacuaagcgagcacuguccaacaacaccagcuccucuccccagccaaagaagaaaccacuggauggagaauauuucacccuucagauccgugggcgugagcgcuucgagauguuccgagagcugaaugaggccuuggaacucaaggaugcccaggcugggaaggagccaggggggagcagggcucacuccagccaccugaaguccaaaaagggucagucuaccucccgccauaaaaaacucauguucaagacagaagggccugacucagacugacauucuccacuucuuguuccccacugacagccucccacccccaucucucccuccccugccauuuuggguuuugggucuuugaacccuugcuugcaauaggugugcgucagaagcacccaggacuuccauuugcuuugucccggggcuccacugaacaaguuggccugcacugguguuuuguuguggggaggaggauggggaguaggacauaccagcuuagauuuuaagguuuuuacugugagggauguuugggagauguaagaaauguucuugcaguuaaggguuaguuuacaaucagccacauucuagguaggggcccacuucaccguacuaaccagggaagcugucccucacuguugaauuuucucuaacuucaaggcccauaucugugaaaugcuggcauuugcaccuaccucacagagugcauugugaggguuaaugaaauaauguacaucuggccuugaaaccaccuuuuauuacauggggucuagaacuugacccccuugagggugcuuguucccucucccuguuggucgguggguugguaguuucuacaguugggcagcugguuagguagagggaguugucaagucucugcuggcccagccaaacccugucugacaaccucuuggugaaccuuaguaccuaaaaggaaaucucaccccaucccacacccuggaggauuucaucucuuguauaugaugaucuggauccaccaagacuuguuuuaugcucagggucaauuucuuuuuucuuuuuuuuuuuuuuuuucuuuuucuuugagacugggucucgcuuuguugcccaggcuggaguggaguggcgugaucuuggcuuacugcagccuuugccuccccggcucgagcaguccugccucagccuccggaguagcugggaccacagguucaugccaccauggccagccaacuuuugcauguuuuguagagauggggucucacaguguugcccaggcuggucucaaacuccugggcucaggcgauccaccugucucagccucccagagugcugggauuacaauugugagccaccacguccagcuggaagggucaacaucuuuuacauucugcaagcacaucugcauuuucaccccacccuuccccuccuucucccuuuuuauaucccauuuuuauaucgaucucuuauuuuacaauaaaacuuugcugccaaaaaaaaaaaaaaaaaaaa

SEQ ID NO: 36 - RNA, 22 nt, Homo sapiens
cccugcccucaacaagauguuu

SEQ ID NO: 37 - RNA, 112 nt, Artificial Sequence
  Anit-p53 asymmetric siRNA molecule
aagauguuuuuucuauucgaccuucagaagagggucgaauagaaaaaacaucuuguugagggcaggguuguagguggcauuucagaagagaugccaccuacaacccugcccu

SEQ ID NO: 38 - RNA, 92 nt, Artificial Sequence
  Anit-p53 asymmetric shRNA molecule
aagauguuuuugcgcgugguagcaaaaacaucuuguugagggcaggguuguagguggcauuucagaagagaugccaccuacaacccugcccu